(19) United States
(12) Patent Application Publication
(10) Pub. No.: US 2024/0254134 A1
(43) Pub. Date: Aug. 1, 2024

(54) AGELASTATIN A DERIVATIVES AND RELATED METHODS

(71) Applicants: Kevin SHUFORD, Waco, TX (US); Haoran XUE, Waco, TX (US); Emviaee CALIXTE, Waco, TX (US); Kenneth HULL, Waco, TX (US); Morgan JOUANNEAU, Waco, TX (US); ROMO, Daniel, Waco, TX (US)

(72) Inventors: Daniel Romo, Waco, TX (US); Kevin Shuford, Waco, TX (US); Haoran Xue, Waco, TX (US); Emvia Calixte, Waco, TX (US); Kenneth Hull, Waco, TX (US); Morgan Jouanneau, Waco, TX (US)

(73) Assignee: Baylor University, Waco, TX (US)

(21) Appl. No.: 18/560,292

(22) PCT Filed: May 11, 2022
(86) PCT No.: PCT/US2022/028759

§ 371 Date: Nov. 10, 2023

Related U.S. Application Data
(60) Provisional application No. 63/187,297, filed on May 11, 2021.

(51) Int. Cl. C07D 487/14; A61K 31/4985

(52) U.S. Cl. CPC C07D 487/14; A61K 31/4985

(57) ABSTRACT

Agelastatin compounds, methods for making agelastatin compounds, and methods for using agelastatin compounds.

### CROSS-REFERENCE TO RELATED APPLICATION

This application claims the benefit of U.S. Application No. 63/187,297, filed May 11, 2021, expressly incorporated herein by reference in its entirety

### STATEMENT OF GOVERNMENT LICENSE RIGHTS

This invention was made with government support under Grant Nos. R37 GM052964 and R35 GM134910 awarded by the National Institutes of Health. The government has certain rights in the invention.

### BACKGROUND

Agelastatin A (AglA, FIG. 1, 1) is one of the most prominent members of the pyrrole-2- aminoimidazole (P-2-AI) family of marine alkaloids owing to its unique structure and broad spectrum of biological activities leading to great interest from both synthetic chemists and biologists. Isolated by Pietra in 1993, this tetracyclic marine alkaloid demonstrated therapeutic potential as a drug lead in the treatment of a variety of cancers including leukemia, breast, lung and glioblastoma. In one study, AglA demonstrated inhibition of osteopontin (OPN, encoded by SPP1), whose overexpression is believed to be associated with neoplastic transformation, cancer progression, and metastasis in a variety of cancers. Furthermore, because of its excellent brain blood barrier penetration, AglA is particularly attractive for the treatment of brain tumors, as OPN is also significantly expressed by primary brain tumors such as glioblastoma multiforme, astrocytoma, and primary central nervous system (CNS) lymphoma. In addition, AglA also inhibits the expression of the glycogen synthase kinase-3β.

Not surprisingly, since its discovery, extensive research has been devoted to the synthesis of AglA and development of a structure-activity-relationship (SAR) profile toward cancer cell lines. A number of total or formal syntheses have been reported featuring a number of elegant synthetic strategies. These studies have led to an extensive understanding of structural requirements for bioactivity and development of several novel derivatives with improved potency. Recently, the cellular target of AglA has been reported, and a working mechanism was elucidated to account for the potent anticancer effects. In HeLa cells, AglA binds to the peptidyl transfer center (PTC) of the ribosome, leading to protein synthesis inhibition and ultimately apoptosis. Moreover, the X-ray structure of the complex of AglA with the S80 subunit of the yeast ribosome opened the possibility of designing novel drug leads based on AglA. In particular, the X-ray structure revealed a number of key hydrogen bonding and n-n stacking interactions and a rare halogen-n interaction. A biomimetic synthesis of AglA has also been described which led to a concise entry to this class of alkaloids and supported a proposed biosynthesis from an acyclic precursor.

Despite the advances in the syntheses of agelastatin derivatives, a need exists for new synthetic methods for making agelastatin derivatives and new agelastatin derivatives having improved therapeutic properties. The present disclosure seeks to fulfill these needs and provides further related advantages.

### SUMMARY

The present disclosure provides agelastatin compounds (e.g., agelastatin A derivatives) and methods for making and using the compounds.

In one aspect, the disclosure provides 7-hydroxy agelastatin compounds having formula (I):

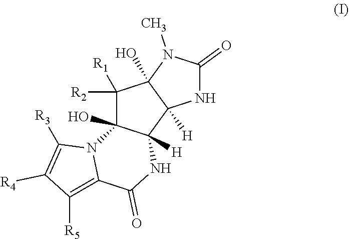

or a stereoisomer, racemate, or a pharmaceutically acceptable salt thereof,
wherein
R₁ is selected from the group consisting of H, F, Cl, and Br;
R₂ is selected from the group consisting of H, F, Cl, and Br;
R₃ is selected from the group consisting of Br, CF₃, SF₅, SO₂CF₃, SO₂CH₃, CN, and NO₂;
R₄ is selected from the group consisting of H, OH, F, Cl, Br, and CN; and
R₅ is H.

In another aspect, the disclosure provides a method for making a compound of formula (I). In certain embodiments, the method comprises converting a compound of formula (A) to a compound of formula (I) via a compound of formula (B):

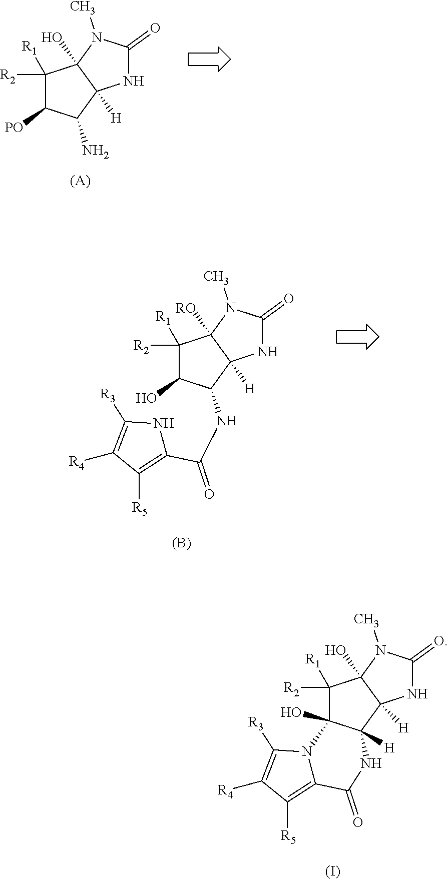

In a further aspect, the disclosure provides a pharmaceutical composition, comprising a 7-hydroxy agelastatin compound as described herein, or a stereoisomer, racemate, or pharmaceutically acceptable salt thereof, and a pharmaceutically acceptable carrier.

In another aspect of the disclosure, a method for treating a cancer is provided. In certain embodiments, the method comprises administering a therapeutically effective amount of a 7-hydroxy agelastatin compound as described herein, or a stereoisomer, racemate, or pharmaceutically acceptable salt thereof, to a subject in need thereof.

In further aspect of the disclosure, a method for inhibiting protein synthesis through interactions with the peptidyl transferase center of a ribosome in a subject is provided. In certain embodiments, the method comprises administering to a subject an effective amount of a 7-hydroxy agelastatin compound as described herein, or a stereoisomer, racemate, or pharmaceutically acceptable salt thereof, in an amount effective to inhibit protein synthesis through interactions with the peptidyl transferase center of a ribosome.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 illustrates the structure of agelastatins (AglA) A-E and an unnatural derivative, 7-hydroxy AglA (7a) revealing hidden pseudo C2-symmetry of a bis-carbinolamine, bis-hydroxy cyclopentane core.

FIG. 2A illustrates the docking of 7-hydroxy AglA (7a) with the X-ray structure of the AglA-yeast 80S ribosome complex using MOE. Full view of binding site (foreground, rRNA; background, protein) showing a possible but weak (3.52 Å) hydrogen bond between the C7-hydroxyl and U2875.

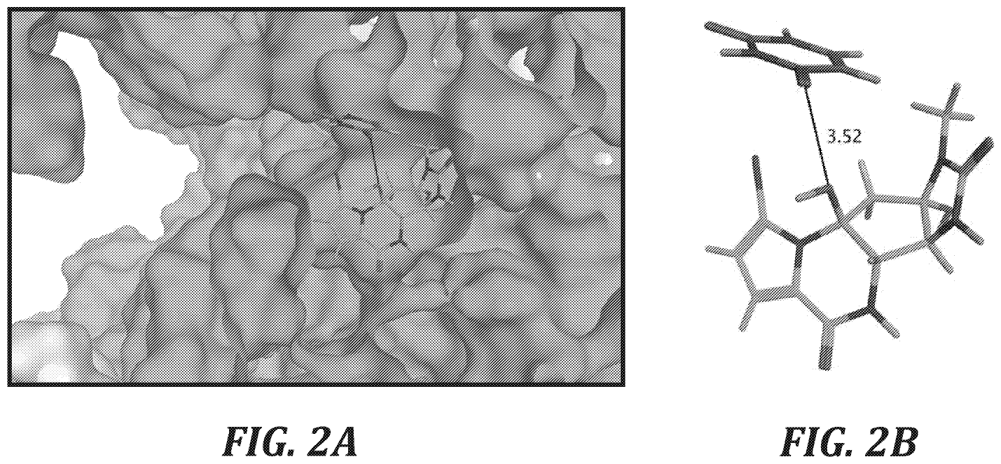

FIG. 2B is an isolated view of 7-hydroxy AglA (7a) and the pyrimidinedione of U2875.

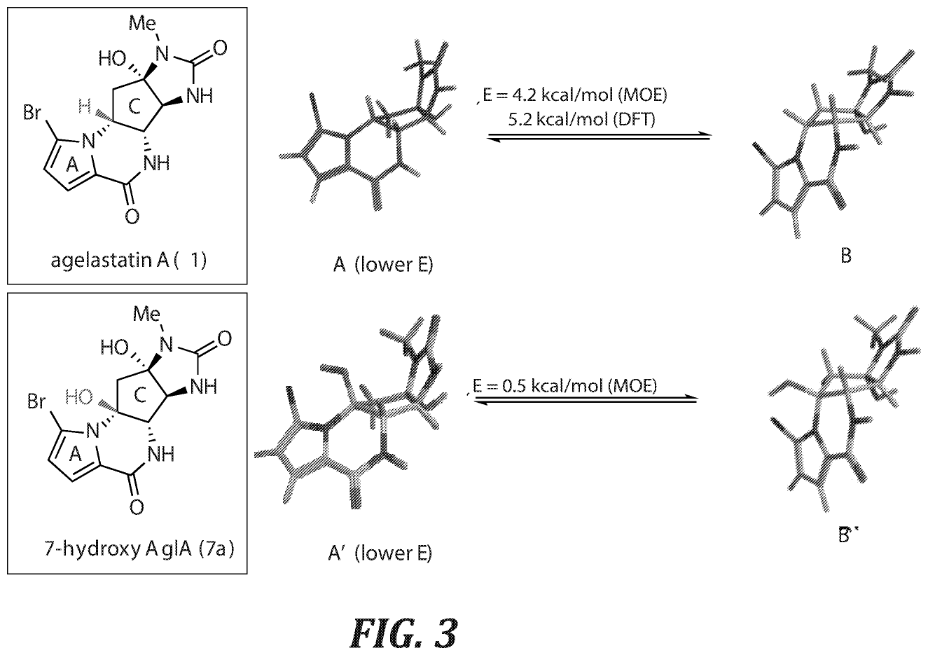

FIG. 3 schematically illustrates conformational searching of AglA and 7-OH AglA by MOE and DFT calculations.

FIG. 4 is a schematic illustration of retrosynthetic analysis of AglA (1) based on a hidden C2 symmetry element, upon addition of a C7-hydroxyl, enabling late-stage pyrrole variation.

FIG. 5 is a schematic illustration of stability studies of pyrrole-derived carbinolamines 18a-18c.

FIG. 6 is a schematic illustration of the synthesis of dibromo 7-hydroxy AglA 24 (inset: X-ray structure of bis-carbinolamine 22).

FIG. 7 is a schematic illustration of an attempted access to the AglA core structure through an intramolecular Mitsunobu reaction.

FIG. 8 is a schematic illustration of the synthesis of AglA via base-promoted aza-Michael ring closure.

FIG. 9 is a schematic illustration of the synthesis of 13-nitro AglA (37).

FIG. 10 is a table summarizing cytotoxicity (EC50, μM) of AglA derivatives against various cancer cell lines.

FIG. 11 illustrates conformational searching of 7-OH des-bromo AglA (24) by MOE and subsequent DFT calculations to determine relative energies.

FIG. 12 is a schematic illustration of a retrosynthetic analysis for introducing representative substituent R₁ and R₂ into agelastatin A compounds.

### DETAILED DESCRIPTION

The present disclosure provides agelastatin compounds (e.g., agelastatin A derivatives) and methods for making and using the compounds.

In one aspect, the disclosure provides 7-hydroxy agelastatin compounds having formula (I):

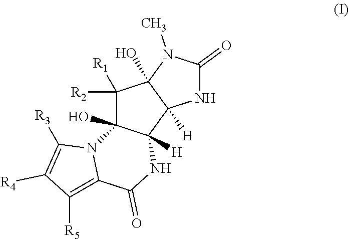

or a stereoisomer, racemate, or a pharmaceutically acceptable salt thereof,
wherein
R₁ is selected from the group consisting of H, F, Cl, and Br;
R₂ is selected from the group consisting of H, F, Cl, and Br;
R₃ is selected from the group consisting of Br, CF₃, SF₅, SO₂CF₃, SO₂CH₃, CN, and N₀₂;
R₄ is selected from the group consisting of H, OH, F, Cl, Br, and CN; and
R₅ is H.

In one embodiment, the disclosure provides a compound of formula (I), wherein R₁, R₂, R₄, and R₅ are H and R₃ is Br.

In another embodiment, the disclosure provides a compound of formula (I), wherein R₁, R₂, and R₅ are H and R₃ and R₄ are Br.

In a further embodiment, the disclosure provides a compound of formula (I), wherein R₁-R₅ are hydrogen.

In others embodiments, the disclosure provides a compound of formula (I), wherein R₁ and R₂ are independently selected from hydrogen and fluoro, hydrogen and chloro, or hydrogen and bromo, where R₃-R₅ are hydrogen. In certain of these embodiments, R₁ is hydrogen and R₂ is fluoro, and in other of these embodiments, R₁ is fluoro and R₂ is hydrogen (i.e., both disasteromers).

In another aspect, the disclosure provides a method for making a compound of formula (I). The methods described herein provide for the introduction of substituents R₁-R₅ into the agelastatin scaffold.

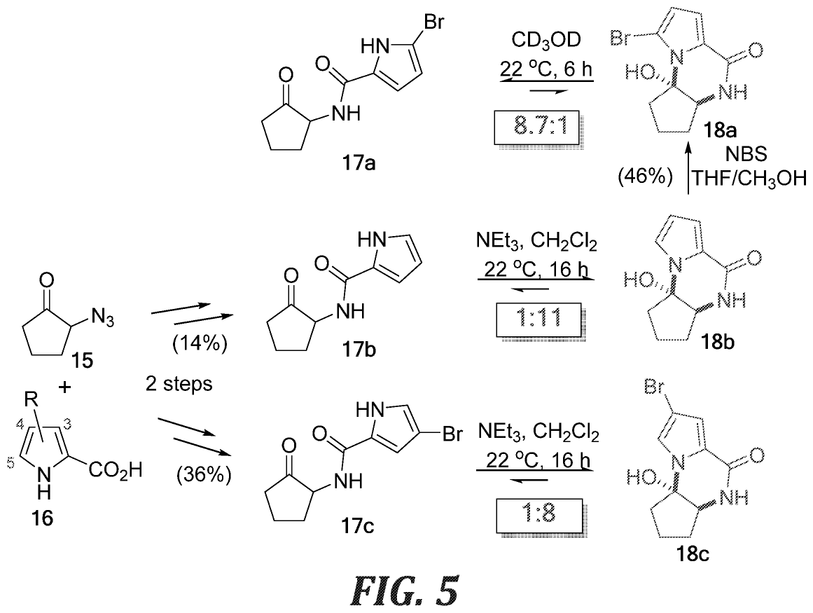

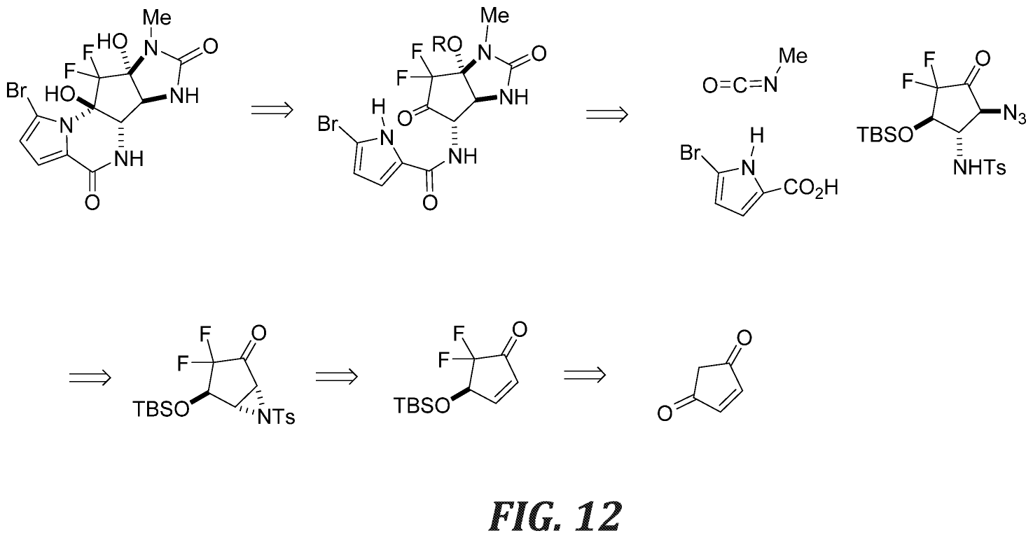

Substituents R₁ and R₂ may be incorporated into the product agelastatin by elaboration of furan (e.g., 15) or cyclopentanone (e.g., 12-14) intermediates. See FIGS. 4-6 and 12. A retrosynthetic analysis for the introduction of fluoro atoms at R₁ and/or R₂ is shown in FIG. 12.

The introduction of mono or dihalogens at R₁ and/or R₂ including fluoro, chloro, or bromo is accomplished as shown in FIG. 12 by halogenation of 4-cylopentene-1,3-dione, or a more advanced cyclopentanone as shown in the syntheses described herein, with an electrophilic source of fluorine like Selectfluor or in the case of chloro or bromo, N-bromosuccinimide, N-chlorosuccinimide, chlorine (Cl₂), bromine (Br₂), or other electrophilic sources of Cl or Br. For example, for the fluorinated derivatives, the dione is treated with a base (e.g., sodium hydride, lithium diisopropylamide) to form the enolate and then reacted with Selectfluor to introduce one or two fluoro-groups.

The present disclosure provides monohalo diastereomers:

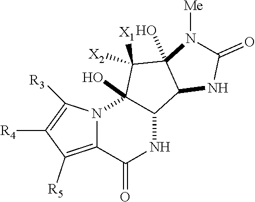

wherein
(1) X₁ is F and X₂ is H;
(2) X₁ is H and X₂ is F;
(3) X₁ is Cl and X₂ is H;
(4) X₁ is H and X₂ is Cl;
(5) X₁ is Br and X₂ is H; and
(6) X₁ is H and X₂ is Br;
and R₃-R₅ are as described above.

Substituents R₃, R₄, and R₅ may be incorporated into the product agelastatin by elaboration of pyrrole (e.g., 11, 16) intermediates. See FIGS. 4-6, 8, and 12.

Details of synthetic schemes are shown in FIGS. 4-6, 8, 9, and 12 and described below in the Experimental Procedures.

In certain embodiments, the method comprises converting a compound of formula (A) to a compound of formula (I) via a compound of formula (B):

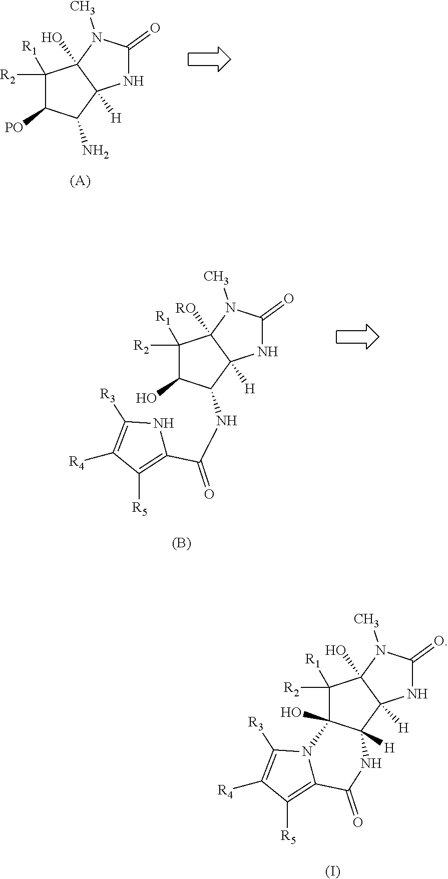

In certain embodiments of the method, the compound of formula (A) is reacted with

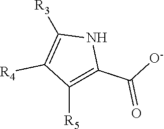

to provide amide (a):

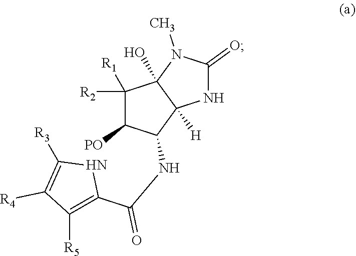

converting amide (a) to the compound of formula (B) followed by ring closure to provide 7-hydroxy compound (b):

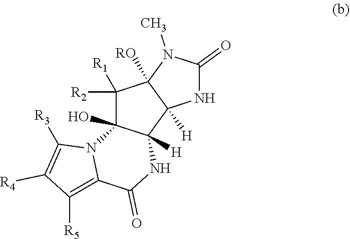

and
converting 7-hydroxy compound (b) to the compound of formula (I) by treatment with aqueous acid,
wherein
P is an alcohol protecting group;
R is a methyl group;
R₁ is selected from the group consisting of H, F, Cl, and Br;
R₂ is selected from the group consisting of H, F, Cl, and Br;
R₃ is selected from the group consisting of Br, CF₃, SF₅, SO₂CF₃, SO₂CH₃, CN, and NO₂;
R₄ is selected from the group consisting of H, OH, F, Cl, Br, and CN; and
R₅ is H.

In a further aspect, the disclosure provides a pharmaceutical composition, comprising a 7-hydroxy agelastatin compound as described herein, or a stereoisomer, racemate, or pharmaceutically acceptable salt thereof, and a pharmaceutically acceptable carrier.

In another aspect of the disclosure, a method for treating a cancer is provided. In certain embodiments, the method comprises administering a therapeutically effective amount of a 7-hydroxy agelastatin compound as described herein, or a stereoisomer, racemate, or pharmaceutically acceptable salt thereof, to a subject in need thereof. In certain embodiments, the cancer is breast cancer (triple negative breast cancer or estrogen receptor positive breast cancer) or glioblastoma.

In further aspect of the disclosure, a method for inhibiting protein synthesis through interactions with the peptidyl transferase center of a ribosome in a subject is provided. In certain embodiments, the method comprises administering to a subject an effective amount of a 7-hydroxy agelastatin compound as described herein, or a stereoisomer, racemate, or pharmaceutically acceptable salt thereof, in an amount effective to inhibit protein synthesis through interactions with the peptidyl transferase center of a ribosome.

As noted above, the present disclosure provides agelastatin A (AglA) derivatives having improved solubility and a synthetic strategy that is more readily amenable to varying the pyrrole moiety to further probe n-n interactions led to the hidden C2-symmetry based strategy described herein that leads to the conception of 7-hydroxy AglA (7a) and related derivatives as a synthetic target.

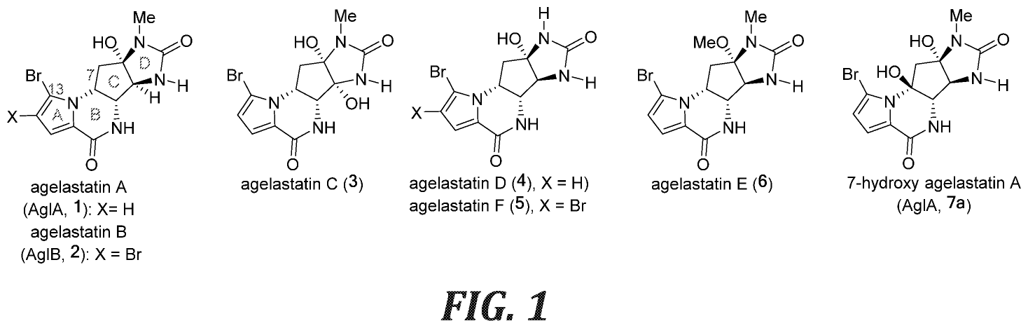

Structurally, AglA bears four nitrogen atoms attached to the central C ring in a syn-anti-syn relationship. We recognized that addition of a C7-hydroxyl group would impart pseudosymmetry to AglA leading to a second carbinolamine, derived from addition of a pyrrole nitrogen to a pendant ketone leading to a hydroxyl-substituted dihydropyrazinone (FIG. 1, 7a). We surmised that addition of an additional hydroxyl group would also impart greater water solubility relative to AglA (clogP: 7-OH AglA (7a), −2.13; AglA (1), −1.23) while also potentially leading to an additional hydrogen bond at the binding site. We docked 7-OH AglA into the X-ray structure of the ribosome-AglA complex (McClary, B.; Zinshteyn, B.; Meyer, M.; Jouanneau, M.; Pellegrino, S.; Yusupova, G.; Schuller, A.; Reyes, J. C.; Lu, J.; Guo, Z.; Romo, D.; Yusupov, M., Green, R.; Liu, J. O. Cell Chem. Bio. 2017, 24, 605-613) using Molecular Operating Environment (MOE) software and found a potential weak (extended, 3.52 Å) hydrogen bond that could be formed between the pyrimidinedione of uracil 2875 (U2875) and the C7-OH in a low energy pose that resembles that of AglA.

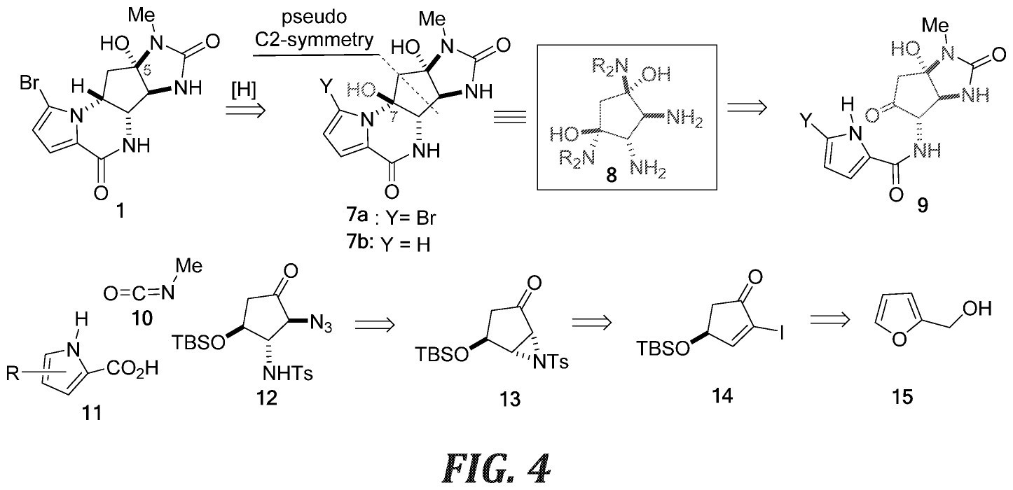

We also wanted to ensure that introduction of the C7-hydroxyl would not significantly impact the overall topology of AglA and ensure that low energy conformations corresponding to AglA were also attainable by 7-OH AglA. Molecular dynamics and minimization in MOE and also Density Functional Theory (DFT) calculations for both AglA and 7-OH AglA showed two similar low energy conformers (A/B and A′/B′) differing only by the envelope conformations adopted by the cyclopentane ring (C-ring) of these otherwise quite rigid molecules (see FIG. 3). In the case of AglA, the two lowest energy conformers A/B differed by ˜4-5 kcal/mol. A ΔG of ˜0.5 kcal/mol (MOE) at 25° C. for the two conformers of 7-OH AglA A′/B′ suggests that they would both be readily accessible at physiological temperatures with one envelope conformation corresponding to the lower energy conformation of AglA.

These preliminary computational studies supported our described synthetic strategy involving a late-stage pyrrole annulation strategy to more readily vary this region of the molecule and expand the known SAR of the agelastatins. This provided further impetus to explore the synthesis of 7-hydroxy AglA based on the described hidden C2-symmetry element found in this targeted derivative and forms the basis of our retrosynthetic strategy (FIG. 4). In addition, this strategy, following reduction of the C7-carbinolamines 7a/7b, could also lead to non-C7-hydroxylated AglA derivatives again with variations in the pyrrole ring. The C7-carbinolamine would be derived from intramolecular cyclization of the pyrrole NH of ketone precursor 9. Bicyclic urea 9 could in turn be synthesized from three fragments: pyrrole 11, azide 12 and N-methylisocyanate (10) through acylation with the isocyanate followed by cyclization onto the ketone. The azide 12 could be introduced by ring cleavage of the aziridine 13, which in turn would be derived from the known cyclopentenone iodide 14, readily available from furfural by a reported procedure ((a) Saitman, A.; Theodorakis, E. A. Org. Lett. 2013, 15, 2410-2413. (b) Yang P.; Yao M.; Li J.; Li Y.; Li A. Angew. Chem. Int. Ed. 2016, 55, 6964-6968. (c)Truax, N.J.; Ayinde, S.; Van, K.; Liu, J. O.; Romo, D. Org. Lett. 2019, 21, 7394-7399).

Carbinolamines are theoretically formed reversibly and can exist in equilibrium with their aldehyde and amine components. Pyrrolocarbinolamines, which are formed by nucleophilic addition of a pyrrole to aldehydes or ketones are reasonably stable and can be purified and isolated, serving as aldehyde protecting groups, but can also be reverted to their carbonyl pyrrole precursors upon treatment with base. It is well known that the C5-carbinolamine of AglA, leading to a hydroxy imidazolidinone exists primarily in the ring-closed form. However, we were unsure of the same equilibrium for the proposed hydroxy dihydropyrazinone 7 derived from cyclization of the pyrrole nitrogen addition onto the pendant ketone. To address this question, we initially performed model studies to probe the stability of such keto pyrrole-derived carbinolamines leading to dihydropyrazinones.

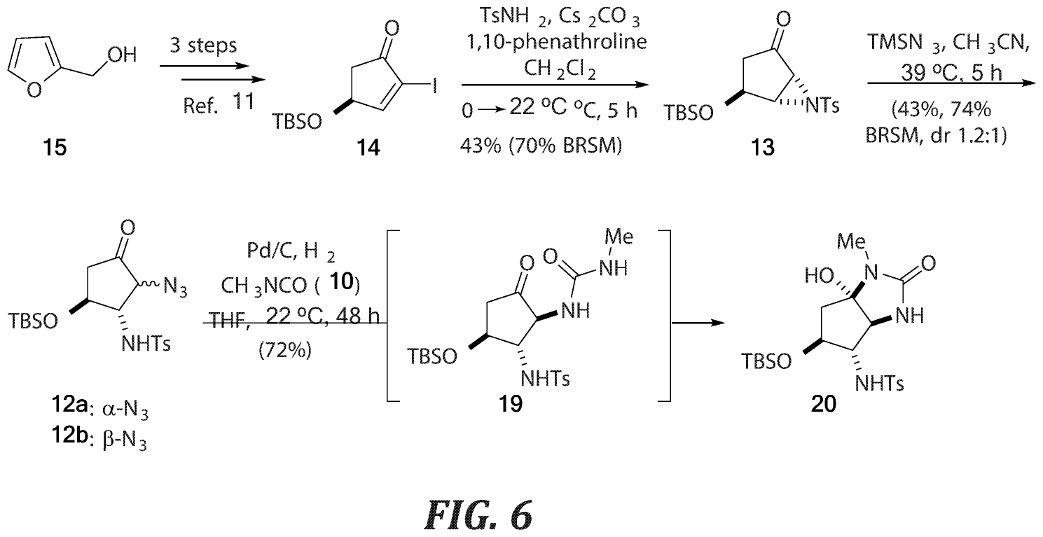

We synthesized pyrrole carboxylic amides 17a-c to study the equilibrium between the corresponding carbinolamines and pyrrolo ketones (FIG. 5). Reduction of 2-azidocyclopentanone and condensation with pyrrole-1H-carboxylic acid gave amide 17b. Upon treatment of ketone 17b with Et₃N, cyclization gave the corresponding carbinolamine 18b in 73% yield (>19:1 dr, 600 MHz ¹H NMR). Carbinolamine 18b was very stable, could be purified by silica gel chromatography, and was stable in CD₃OD for several days. When resubjected to NEt₃/CH₂Cl₂ at ambient temperature (22° C.) for 16 h, only a small amount of the ring-opened keto pyrrole 17b was generated leading to an equilibrium ratio of 11:1 (18b/17b) favoring the cyclized form as judged by ¹H NMR. Likewise, the 4-bromopyrrole derived carbinolamine 18c was stable under neutral conditions (e.g. in CD₃OD), but led to a mixture of 18c and 17c in an 8:1 ratio in NEt₃/CH₂Cl₂ at 22° C. again favoring the cyclized form. In contrast, the 5-bromo pyrrole analog 17a, that most closely mimics the targeted 7-hydroxy AglA, disfavored the closed form 18a. Not only did the precursor keto pyrrole 17a not cyclize to 18a under similar conditions as for 17b and 17c, even if the cyclic carbinolamine 18a was targeted through bromination of carbinolamine 18b, the brominated adduct 18a opened rapidly in CD₃OD forming a mixture of 17a/18a in an 8.7:1 ratio, favoring the keto pyrrole 17a. Overall, these results suggested that substituents present on C5 of the pyrrole ring disfavor the cyclic carbinolamine, thus we initially pursued the non-brominated C7-OH AglA 7b.

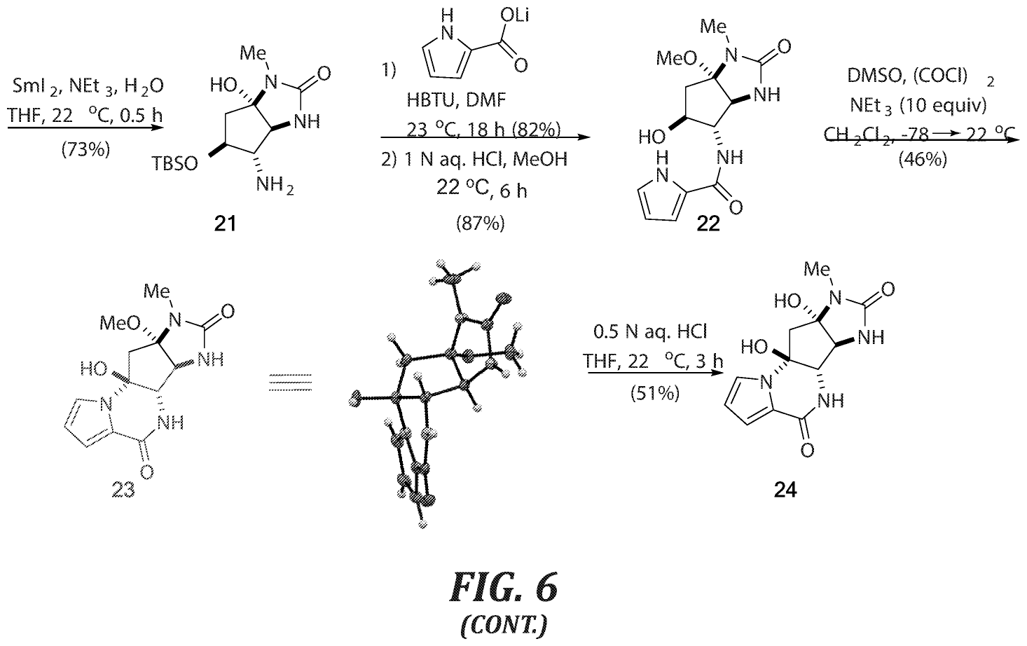

Synthesis of racemic 7-hydroxy-13-des-bromo AglA (24) commenced with aziridination of known iodide 14, available in 3 steps from furfuryl alcohol (FIG. 6) ((a) Saitman, A.; Theodorakis, E. A. Org. Lett. 2013, 15, 2410-2413. (b) Yang P.; Yao M.; Li J.; Li Y.; Li A. Angew. Chem. Int. Ed. 2016, 55, 6964-6968. (c)Truax, N.J.; Ayinde, S.; Van, K.; Liu, J. O.; Romo, D. Org. Lett. 2019, 21, 7394-7399). Aziridination with p-toluenesulfonamide under basic conditions installed the aziridine 13 by the method of Maycock (Silva, S.; Rodrigues, P.; Bento, I.; Maycock, C. D. J. Org. Chem. 2015, 80, 3067-3074). Aziridine cleavage with azide anion was studied next however instability of both the aziridine and the derived azide to both acidic and basic conditions was observed.

Following extensive experimentation, trimethylsilyl azide was found to be optimal for aziridine ring opening. This led to the azidocyclopentanone 12 as a mixture of diastereomers (43%, dr 1.2:1), likely due to an unselective a-protonation of the intermediate silyl enol ether. The desired anti-diastereomer 12b was isolated by column chromatography, while the undesired diastereomer 12a could be re-equilibrated by subjecting to 4 Å molecular sieves in acetonitrile for 6 days leading to an ˜1:1 ratio and isolation of additional quantities of the desired anti-diastereomer. Hydrogenation of azide 12a in the presence of N-methyl isocyanate (10) to furnish the bicyclic intermediate 20 in a single step through known cyclization to the cyclic urea. To introduce the pyrrole moiety, the N-tosyl group was cleaved using SmI₂/H₂O/NEt₃ (Ankner T.; Hilmersson, G. Org. Lett. 2009, 11, 503-506) providing the primary amine 21. Subsequent amide coupling with the lithium carboxylate of pyrrole 2-carboxylic acid was initially problematic and could not be pushed to completion due to the insolubility of primary amine 21 which was found to be only partially soluble in typical organic solvents for amide couplings (e.g., CH₂Cl₂, DMF).

Ultimately, it was found that a suspension of the amine 21 in DMF would dissolve completely upon heating to ˜100° C. providing a homogenous solution. After cooling to ambient temperature, coupling proceeded smoothly to afford the desired amide 22 in 82% yield.

Deprotection of the TBS ether was achieved with HCl in MeOH which also served to mask the hemiaminal as the methoxy aminal 22. Swern oxidation led to an intermediate ketone, which in the presence of excess triethylamine, cyclized to deliver the carbinolamine 23 directly in 46% yield. This is consistent with our model studies (cf FIG. 5, 18b) which suggested this equilibrium would favor the cyclic form of the C7-carbinolamine and this was further confirmed by X-ray crystallography of carbinolamine 23 (inset, FIG. 6). Interestingly, the conformation in the solid state corresponds to the lower energy conformation found through conformational searching (vide supra). Hydrolysis of the methoxyaminal 15 was achieved under mild conditions to deliver the targeted 7-hydroxy-13-des-bromo AglA (24), which was found to be quite stable as determined by ¹H NMR when stored in CD₃OD or DMSO-d₆ at ambient temperature (22° C.) for 7 days.

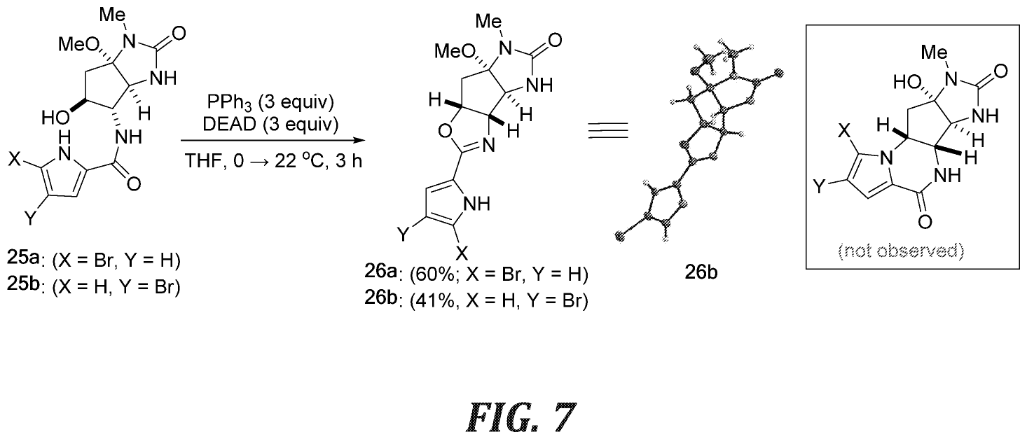

In efforts to convert the mono-carbinolamine 23 to the core structure of AglA, we explored several direct methods; however, direct reduction was not fruitful under a variety of Lewis acid/hydride addition conditions. Alternatively, we considered direct intramolecular stereoinvertive displacement of alcohols 25a and 25b, derived from amine 21 (see 25a,b), through Mitsunobu reaction to form the B ring. However, while cyclization indeed occurred, it was the amide oxygen atom that served as nucleophile leading to oxazolines 26a and 26b as confirmed by X-ray crystallography in the case of oxazoline 26b (FIG. 7).

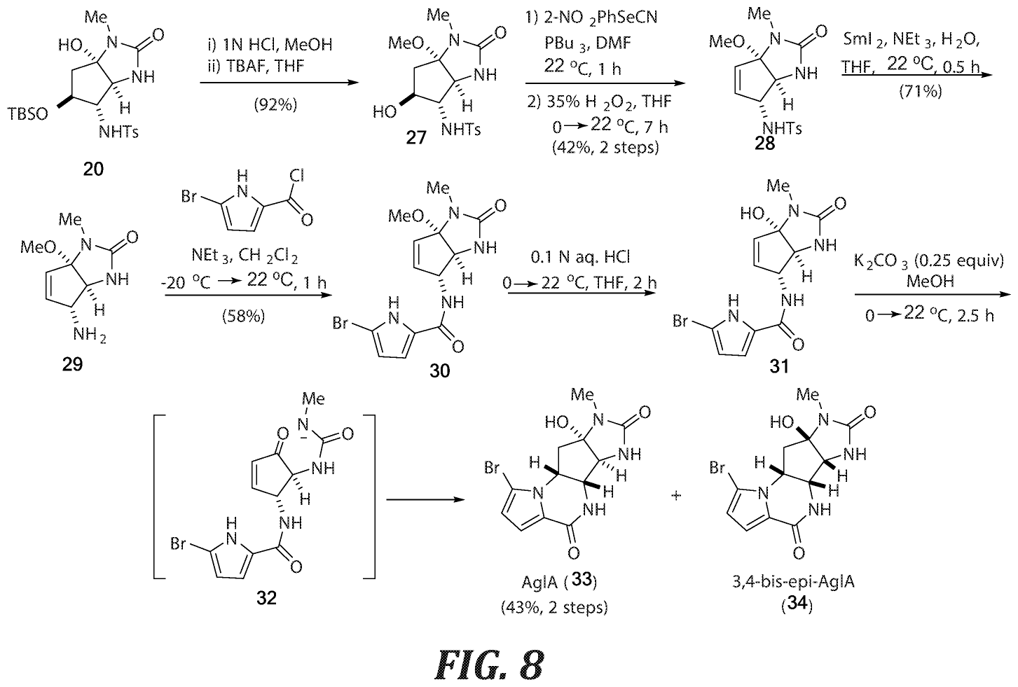

We next considered an alternative strategy to form the B ring that was successfully employed in our previous biomimetic strategy toward AglA (Reyes, J. C. P.; Romo, D. Angew. Chem., Int. Ed. 2012, 51, 6870-6873), namely a 5-exo, aza-Michael ring closure. Attempted dehydration of alcohol 25a to directly install the C6-C7 alkene was very low yielding due to multiple side reactions (not shown). We thus returned to an earlier intermediate, silyl ether 20, and following generation of the methoxy carbinolamine and desilylation, we introduced the required C6-C7 alkene through a Grieco elimination to give cyclopentene 28 in 42% yield for the 2 steps (FIG. 8). Cleavage of the tosyl group with SmI₂ and acylation with the pyrrole acid chloride provided amide 30, which was hydrolyzed to carbinolamine 31 to intercept the same intermediate in our previous biomimetic synthesis of AglA. Carbinolamine was subjected to mild heating with silica gel to initiate the aza-Michael addition and this was again successful to deliver AglA.

However, we sought alternative reaction conditions for this transformation since it was not readily scalable. We anticipated that if the concentration of the ring opened C-ring enone of carbinolamine 31 could be achieved under mild basic conditions, this would facilitate the desired aza-Michael reaction. After some experimentation with a variety of bases, we found that a sub-stoichiometric amount of base, namely 0.25 equiv K₂CO₃ in MeOH, furnished AglA (33) in 43% yield (2 steps) along with the known 3,4-bis-epi-AglA (34). The spectra of both products matched that previously reported.

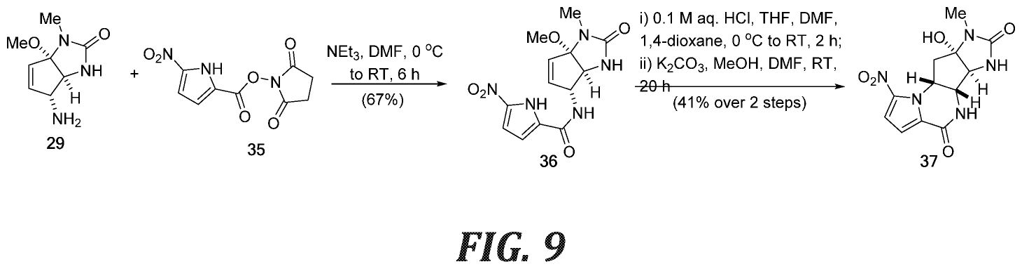

Previous SAR studies of AglA have shown electron withdrawing groups on the pyrrole moiety are beneficial for the potency of derivatives (e.g., Cl, CF₃) (Stout, E. P.; Choi, M. Y.; Castro, J. E.; Molinski, T. F. J. Med. Chem. 2014, 57, 5085-5093. Jouanneau, M.; McClary, B.; Reyes, J. C. P.; Chen, R.; Chen, Y.; Plunkett, W.; Cheng, X.; Milinichik, A. Z.; Albone, E. F.; Liu, Jun O. Bioorg. & Med. Chem. Lett. 2016, 26, 2092-2097). This observation is consistent with the observed n-n stacking observed with nucleotide bases from ribosomal RNA in the X-ray structure of AglA bound to the A-site of the ribosomal peptidyl transfer center. We therefore targeted the electron withdrawing nitro group to demonstrate the utility of this late-stage pyrrole annulation strategy, despite the potential liabilities of the nitro group from a medicinal chemistry perspective. The substrate for the key aza-Michael cyclization was readily prepared from amine 29 in a similar manner as described above for AglA but using the N-hydroxysuccinimide (NHS) ester of the nitro substituted pyrrole 35 to avoid the bis-epimerization at C1,C5 (cf 34) of bicyclic imidazolidinone 29 found to occur readily under even mild acidic conditions (FIG. 9). In a similar fashion, the aza-Michael cyclization proceeded smoothly to furnish 13-nitro AglA (37) in 41% yield over 2 steps. Interestingly, this AglA derivative 27 precipitated as a light-yellow solid from the reaction mixture, and a simple filtration and washing with methanol afforded nitro AglA derivative 37 in pure form.

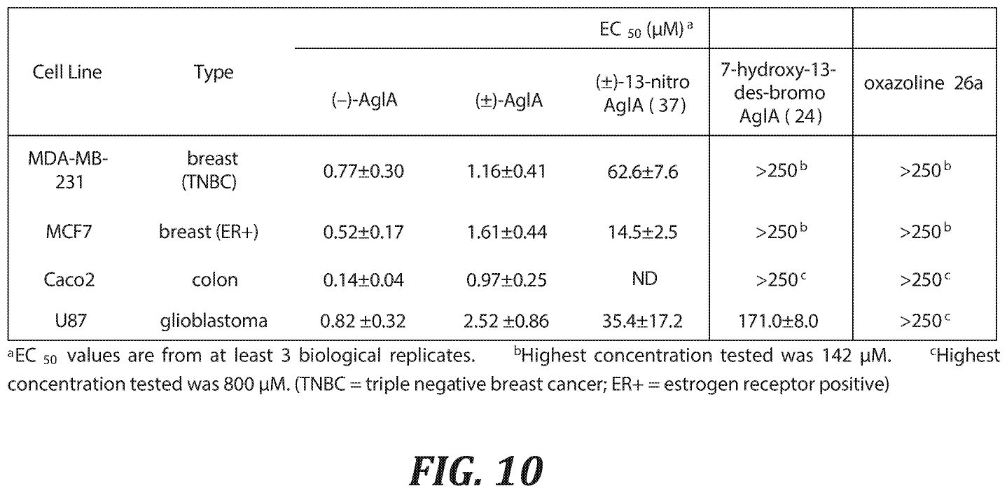

The cytotoxicity of the synthesized novel AglA derivatives was determined against four cancer cell lines in comparison to both (−)-AglA and (±)-AglA (FIG. 10). The bis-carbinolamine 24, lacking the C13-bromo substituent, did not show activity against MCF7, Caco2, and MDA-MB-231 cell lines up to 250 μM. This is not surprising based on the apparent halogen-p interaction observed in the X-ray structure, and the previously demonstrated ˜500X drop in potency of 13-des-bromo AglA HeLa cells compared to AglA. Unfortunately, as suggested by our model studies (vide supra, FIG. 5, 17a), 7-OH AglA (24), bearing the C5-bromo substituent, was found to exist primarily as the ring opened ketopyrrole tautomer which led to instability and thus could not be assayed. However, a measurable EC₅₀ value (171.0±8.0 μM) could be obtained for the bis-carbinolamine 24 against the glioblastoma cell line, U87. The oxazoline 26a showed no activity against any cell line studied.

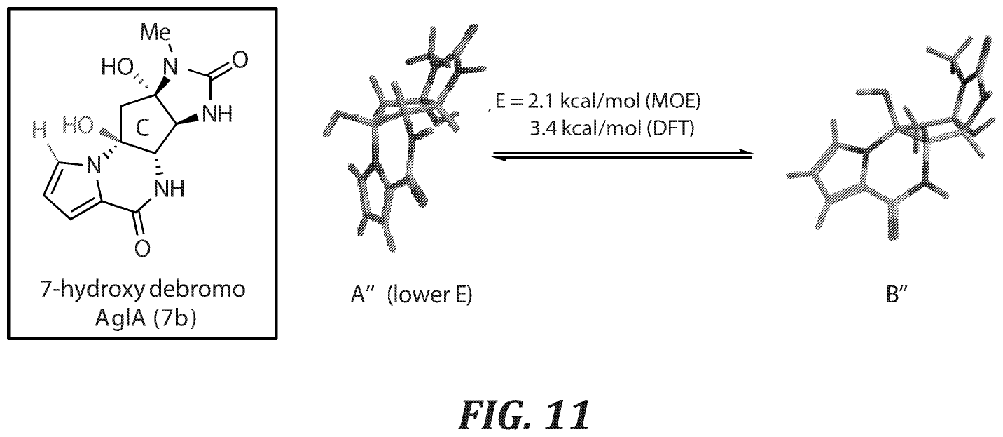

The reduced bioactivity of the des-bromo variant of 7-OH AglA (24) encouraged us to perform a conformational analysis of this derivative (FIG. 11). Of particular interest was a comparison of the C-ring cyclopentane conformational preferences compared to AglA. Indeed, the envelope ring conformer A″ was determined to be of lower energy (ilE=3.4 kcal/mol) than the lowest energy cyclopentyl conformer A of AglA (cf FIG. 3) and thus the lower concentration of the AglA-like envelope conformer may contribute to the reduced cytotoxicity.

The present disclosure provides a novel synthetic strategy based on a hidden symmetry element leading to bis-carbinolamine derivatives of AglA (e.g., C7-hydroxy dibromo AglA (24)) with the important feature of enabling late-stage variations of the pyrrole moiety. This design was guided by the X-ray structure of the AglA-ribosome complex and molecular modeling. While the targeted 7-hydroxy AglA primarily resided in the ring-opened keto pyrrole form was found to be unstable, 7-hydroxy des-bromo AglA could be synthesized and isolated. In addition, a new set of conditions was developed for a final 6-exo-trig, aza-Michael ring closure and this was utilized to access 13-nitro AglA (37). The biological activities of new AglA derivatives were measured against four cancer cell lines and a novel C5-nitro AglA showed activity against all cell lines studied (1.16-35.4 μM) except the colon cancer line, Caco-2. However, 7-OH des-bromo AglA (24), despite missing the critical C5-bromo substituent, did exhibit cytotoxicity toward the glioblastoma cell line tested (U87, EC₅₀=171.0±8.0 μM).

The following examples are provided for the purpose of illustration, not limitation.

### EXAMPLES

### General Information

Unless otherwise described, all non-aqueous reactions were carried out in flame-dried glassware under a N₂ atmosphere. All solvents used were dried through use of an activated molecular sieve-based solvent purification system. Triethylamine was distilled over CaH₂ prior to use. All other commercial reagents were used as received. ¹H NMR spectra were measured at 600 MHz, 500 MHz or 400 MHz, and chemical shifts are reported as 8 values in ppm relative to CDCl₃ (7.26 ppm) or CD₃OD (3.31 ppm). Coupling constants (J) are reported in Hertz (Hz), and multiplicity follows convention. The abbreviations brs (broad singlet), s, d, t, q, p, hept and m (or any combination of these) stand for the resonance multiplicities broad singlet, singlet, doublet, triplet, quartet, pentet, heptet and multiplet. The designation app indicates apparent for splitting that was observed but not necessarily predicted and broad indicates a broadening of a peak possibly due to additional smaller coupling which cannot be discerned. Deuterated solvents (CDCl₃, 77.16 ppm, CD₃OD, 49.00 ppm, or DMSO, 39.52 ppm) served as the internal standard for ¹³C NMR spectra. Flash column chromatography was performed using 60 Å silica gel (Silicycle, 230-400 mesh) as the stationary phase using a gradient solvent system or on an automated flash chromatography system. High resolution mass spectra were obtained at the Mass Spectrometry Center (Baylor University). Thin layer chromatography (TLC) was performed using pre-coated glass-backed TLC plates, Silica Gel F₂₅₄ (Silicycle, 250 μm thickness). Fourier Transformation Infrared (FTIR) spectra were recorded as thin films on NaCl plates. X-ray structures were obtained at the X-ray Diffraction Laboratory at Baylor University.

Abbreviation List

| | |
|---|---|
| (COCl)₂ | Oxalyl chloride |
| DEAD | Diethyl azodicarboxylate |
| DMAP | 4-Dimethylaminopyridine |
| DMF | N,N-Dimethylformaldehyde |
| DMP | Dess-Martin periodinane |
| DMSO | Dimethyl sulfoxide |
| HBTU | Hexafluorophosphate benzotriazole tetramethyl uranium |
| NBS | N-Bromosuccinimide |
| NEt₃ | Triethylamine |
| 2-NO₂PhSeCN | 2-Nitrophenyl selenocyanate PBu₃ Tributylphosphine |
| PPh₃ | Triphenylphosphine |
| SmI₂ | Samarium diiodide |
| TBAF | Tetrabutylammonium fluoride |
| TBSCl | tert-Butyldimethylsilyl chloride |
| THF | Tetrahydrofuran |
| TMSN₃ | Trimethylsilyl azide |
| TsNH₂ | p-Toluenesulfonamide |

### Experimental Procedures

The following provides a description of the synthesis of the compounds described herein. Schematic illustrations of these syntheses are illustrated in FIGS. 4-9.

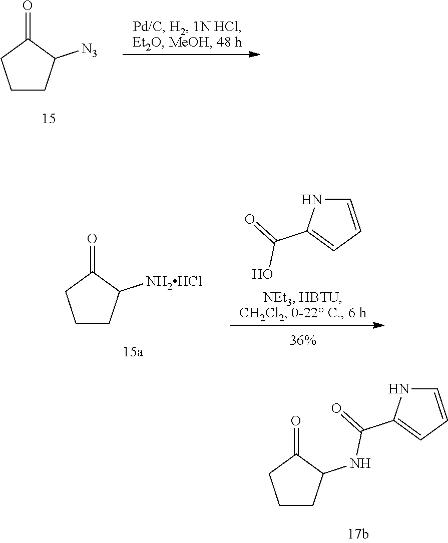

To azide 15 (Dede, D. Regioselective synthesis of functionalized salicylates, isotetronic acids, and alkylidene-isobenzofurans based on one-pot cyclization of enol ethers; Rostock, Germany, 2008) (0.30 g, 2.4 mmol, 1.0 equiv) in methanol (2.4 mL) was added 10% palladium on carbon (133 mg, 0.28 mmol, 0.12 equiv). A H₂ balloon attached to the flask via a 3-way adapter was connected and the flask was sequentially placed under vacuum and backfilled with H₂ (3 times). 1M hydrogen chloride in diethyl ether (4.08 mL, 4.08 mmol, 1.7 equiv) was added and the reaction was stirred for 21 h. A second portion of palladium on carbon (66.5 mg, 0.14 mmol, 0.06 equiv) and methanol (0.6 mL) was added and the reaction was stirred with a new hydrogen balloon for another 17 h. TLC suggested disappearance of starting material, and the mixture was filtered through a cotton plug, washed with methanol and concentrated in vacuo to give the amine hydrogen chloride salt 15a as a black solid (0.3 g) which was used directly in the next step.

To a 10 mL round-bottom flask was charged pyrrole-1H-2-carboxylic acid (214.2 mg, 1.93 mmol, 1.5 equiv) and THF (6.0 mL). To this clear solution was added hexafluorophosphate benzotriazole tetramethyl uranium (HBTU, 731.2 mg. 1.93 mmol, 1.5 equiv) and triethylamine (0.54 mL, 3.87 mmol, 2.0 equiv) at ambient temperature (22° C.), followed by the addition of DNMIF (1.5 mL) to form a colorless clear solution. The mixture was stirred for 1 h and then cooled to 0° C. A solution of 15a (174.3 mg, 1.29 mmol, 1.0 equiv), prepared as described above, N,N-dimethylformaldehyde (3.3 mL) was added, and the reaction was allowed to warm up to ambient temperature (22° C.) by removing ice-water bath. The reaction was stirred for another 6 h and concentrated in vacuo to remove the solvents. The organic residue was purified by MPLC on silica (ethyl acetate/hexane, 0 to 60%) to give the amide 17b (89.0 mg, 36%) as a white solid.

17b: White solid. ¹H NMR (400 MHz, CD₃OD) δ 6.91 (dd, J=2.6, 1.4 Hz, 1H), 6.79 (dd, J=3.8, 1.5 Hz, 1H), 6.16 (dd, J=3.7, 2.5 Hz, 1H), 4.37-4.13 (m, 1H), 2.45-2.33 (m, 2H), 2.32-2.22 (m, 1H), 2.14-2.02 (m, 1H), 2.00-1.80 (m, 2H), two NH protons were not observed; ¹³C NMR (100 MHz, CD₃OD) δ 217.6, 163.5, 126.4, 123.1, 112.0, 110.2, 58.1, 36.7, 30.32, 19.41; IR (thin film, cm⁻¹) 1737, 1626, 1557, 1519, 1129; HRMS (ESI) calcd for C₁₀H₁₃N₂O₂⁺ [M+H]⁺ 193.0972, found 193.0979.

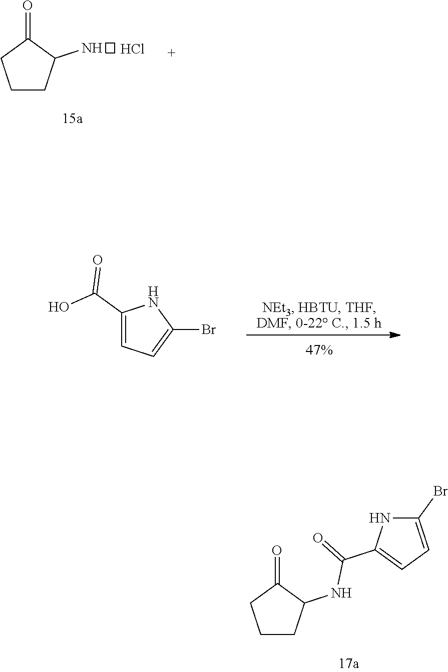 [SMILES: NC1CCCC1=O ; Cl ; O=C(O)c1ccc(Br)[nH]1 ; "47%" ; "NEt3, HBTU, THF,DMF, 0-22° C., 1.5 h" ; O=C(NC1CCCC1=O)c1ccc(Br)[nH]1]

To a 10 mL round-bottom flask was charged 5-bromopyrrole-1H-2-carboxylic acid (118.8 mg, 0.625 mmol, 1.5 equiv) and hexafluorophosphate benzotriazole tetramethyl uranium (HBTU, 237.0 mg, 0.625 mmol, 1.5 equiv). THF (2.0 mL) was added followed by the addition of triethylamine (0.23 mL, 1.67 mmol, 4.0 equiv) at ambient temperature (22° C.). DMF (0.5 mL) was added and the suspension became homogeneous. The mixture was stirred for 1 h and cooled to 0° C. A solution of 15a (56.5 mg, 0.42 mmol, 1.0 equiv)), prepared as described above for 17b, in N,N-dimethylfornaldehyde (1.1 mL) was added, and the reaction was allowed to warm to ambient temperature (22° C.) by removing ice-water bath. The reaction was stirred for another 1.5 h and concentrated in vacuo to remove the solvents. The organic residue was purified by MPLC on silica (ethyl acetate/hexane, 0 to 60%) to give the amide 17a (53.0 mg, 47%) as a white solid.

17a: White solid. ¹H NMR (500 MHz, CD₃OD) δ 6.73 (d, J=3.8 Hz, 1H), 6.13 (d, J=3.8 Hz, 1H), 4.25 (dd, J=11.3, 8.7 Hz, 1H), 2.42-2.31 (m, 2H), 2.31-2.20 (m, 1H), 2.13-2.02 (m, 1H), 1.98-1.81 (m, 2H), two NH protons were not observed; ¹³C NMR (125 MHz, CD₃OD) δ 217.4, 162.3, 128.3, 113.5, 112.5, 104.6, 58.1, 36.6, 30.3, 19.4; IR (thin film, cm⁻¹) 3183, 1744, 1623, 1555, 1455, 1394; HRMS (ESI) calcd. for C₁₀H₁₁⁷⁹BrN₂NaO₂⁺ [M+Na]⁺292.9896, found 292.9901.

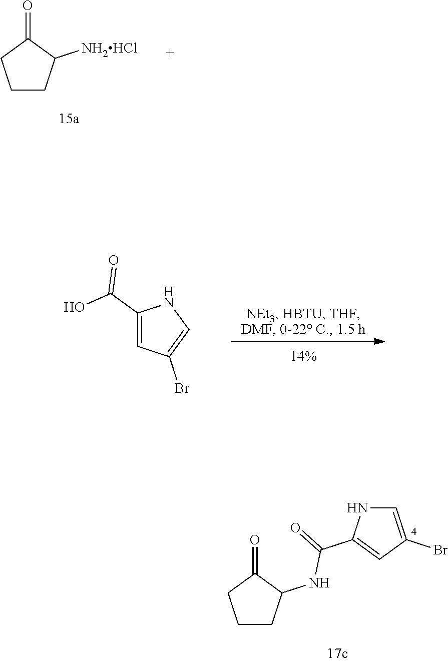

To a 10 mL round-bottom flask was added 4-bromopyrrole-1H-2-carboxylic acid (130.3 mg, 0.69 mmol, 1.5 equiv) and hexafluorophosphate benzotriazole tetramethyl uranium (HBTU, 260.1 mg, 0.69 mmol, 1.5 equiv), followed by the addition of THF (2.0 mL) and triethylamine (0.26 mL, 1.83 mmol, 4.0 equiv) at 22° C. DMF (0.5 mL) was added and the suspension became a clear solution. The mixture was stirred for 1 h and cooled to 0° C. In another 1.5 dram vial, 15a (62 mg, 0.46 mmol, 1.0 equiv) was dissolved in DMF (1.1 mL) and the solution was transferred via cannula to the reaction vessel, and then the reaction was allowed to warm up to ambient temperature (22° C.) by removing ice-water bath. The reaction was stirred for another 1.5 h and concentrated in vacuo to remove the solvents. The organic residue was purified by MPLC on silica (ethyl acetate/hexane, 0 to 60%) to give the amide 17c (17.0 mg, 14%) as an off-white solid.

17c: Off-white solid. ¹H NMR (600 MHz, CD₃OD) δ 6.92 (br t, J=1.3 Hz, 1H), 6.78 (d, J=1.5 Hz, 1H), 4.25 (dd, J=11.4, 8.2 Hz, 1H), 2.41-2.34 (m, 2H), 2.27 (ddd, J=19.7, 11.0, 9.3 Hz, 1H), 2.10 (m, 1H), 2.00-1.84 (m, 2H), one NH protons was not observed; ¹³C NMR (150 MHz, CD₃OD) δ 217.3, 162.3, 127.1, 123.0, 113.5, 97.5, 58.1, 36.6, 30.2, 19.4; IR (thin film, cm⁻¹) 3219, 1746, 1633, 1563, 1523; HRMS (ESI) calcd for C₁₀H ⁷⁹BrN₂O₂[M−H]⁻ 268.9931, found 268.9935.

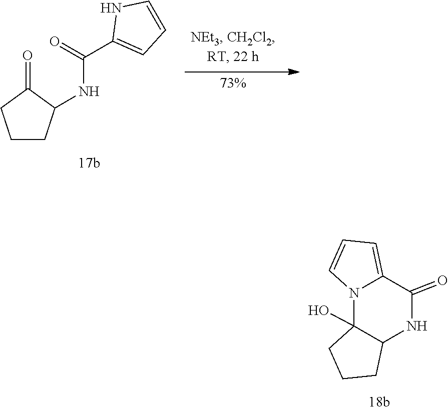 [SMILES: O=C(NC1CCCC1=O)c1ccc[nH]1 ; O=C1NC2CCCC2(O)n2cccc21]

To a stirred solution of amide 17b (13.0 mg, 0.068 mmol, 1.0 equiv) in dichloromethane (1.3 mL) was added triethylamine (28 ul, 0.20 mmol, 3.0 equiv) at ambient temperature (22° C.). The mixture was stirred for 22 h and the reaction was concentrated. The residue was purified by column chromatography (silica, methanol/dichloromethane 0 to 15%) to give cyclic carbinolamine 18b (9.5 mg, 73%) as a white solid.

18b: White solid. ¹H NMR (600 MHz, CD₃OD) δ 7.21 (dd, J=2.7, 1.6 Hz, 1H), 6.88 (dd, J=3.8, 1.6 Hz, 1H), 6.29 (dd, J=3.8, 2.7 Hz, 1H), 3.88 (dd, J=7.2, 5.9 Hz, 1H), 2.32 (ddd, J=13.9, 9.6, 5.7 Hz, 1H), 2.29-2.22 (m, 1H), 2.19 (ddd, J=13.8, 9.5, 6.5 Hz, 1H), 1.87 (m, 1H), 1.78 (m, 1H), 1.67 (m, 1H); OH and NH protons not observed; ¹³C NMR (150 MHz, CD₃OD) δ 161.8, 123.4, 122.3, 115.3, 111.5, 90.5, 62.7, 38.3, 32.1, 20.5; IR (thin film, cm⁻¹) 3272, 1633, 1558, 1458, 1330; HRMS (ESI) calcd for C₁₀H₁₂N₂NaO₂⁺ [M+Na]⁺215.0791, found 215.0801.

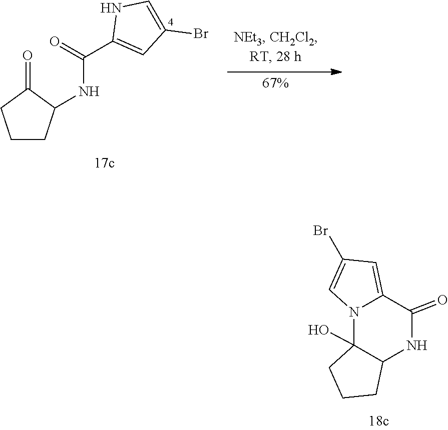 [SMILES: O=C(NC1CCCC1=O)c1cc(Br)c[nH]1 ; O=C1NC2CCCC2(O)n2cc(Br)cc21]

To a stirred solution of amide 17c (6.0 mg, 0.022 mmol, 1.0 equiv) in dichloromethane (0.2 mL) was added triethylamine (9 mL, 0.066 mmol, 3.0 equi) at ambient temperature (22° C.). The mixture was stirred for 28 h, and the reaction was concentrated. The residue was purified by column chromatography (silica, methanol/dichloromethane, 0 to 15%) to isolate the cyclized product 18c (4.0 mg, 67%) as a white solid.

18c: White solid. ¹H NMR (600 MHz, CD₃OD) δ 7.23 (d, J=1.8 Hz, 1H), 6.82 (d, J=1.8 Hz, 1H), 3.88 (dd, J=7.2, 6.0 Hz, 1H), 2.31 (ddd, J=13.7, 9.5, 5.6 Hz, 1H), 2.28-2.22 (m, 1H), 2.19 (ddd, J=13.9, 9.4, 6.5 Hz, 1H), 1.92-1.84 (m, 1H), 1.84-1.73 (m, 1H), 1.68 (m, 1H); ¹³C NMR (150 MHz, CD₃OD) δ 160.4, 124.5, 121.9, 116.4, 99.9, 90.9, 62.6, 38.2, 32.1, 20.5; IR (thin film, cm⁻¹) 3295, 1637, 1553, 1470; HRMS (ESI) calcd for C₁₀H₁₀⁷⁹BrN₂O₂[M−H]⁻ 268.9931, found 268.9935.

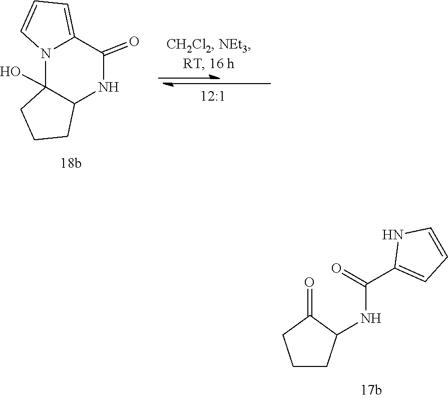 [SMILES: O=C1NC2CCCC2(O)n2cccc21 ; O=C(NC1CCCC1=O)c1ccc[nH]1]

To a stirred solution of carbinolamine 18b (3.0 mg, 0.016 mmol, 1.0 equiv) in dichloromethane (0.32 mL) was added triethylamine (6.6 μl, 0.047 mmol, 3.0 equiv). The reaction was stirred at ambient temperature (22° C.) for 16 h while monitoring by ¹H NMR which showed formation of a mixture of ketone 18b and cyclic carbinolamine 17b in a 12:1 ratio, respectively, and this ratio remained unchanged over this time period.

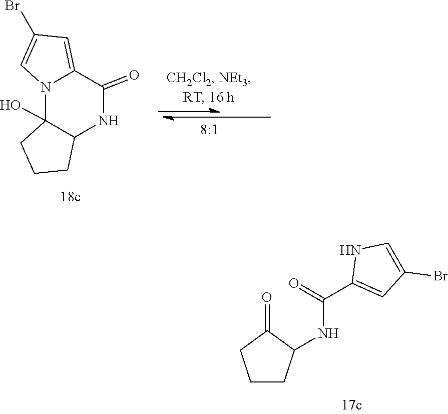 [SMILES: O=C1NC2CCCC2(O)n2cc(Br)cc21 ; O=C(NC1CCCC1=O)c1cc(Br)c[nH]1]

To a stirred solution of carbinolamine 18c (1.2 mg, 0.0044 mmol, 1.0 equiv) was added a solution of triethylamine in dichloromethane (0.1 mL, the solution was made by mixing 19 μl triethylamine and 1.0 mL dichloromethane). The reaction was stirred for 16 h while monitoring by ¹H NMR which showed formation of a mixture of the ketone 18c and cyclic carbinolamine 17c in a 8:1 ratio, respectively, and this ratio remained unchanged over this time period.

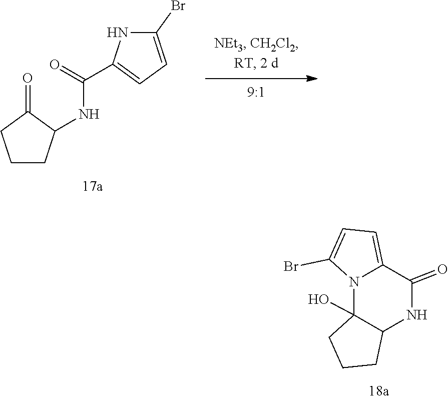 [SMILES: O=C(NC1CCCC1=O)c1ccc(Br)[nH]1 ; O=C1NC2CCCC2(O)n2c(Br)ccc21]

To a 1.0 dram vial was charged 17a (2.7 mg, 0.01 mmol, 1.0 equiv) and a solution of triethylamine in dichloromethane (0.23 mL, 0.03 equiv, 3.0 equiv. The solution was made by mixing 19 μl triethylamine and 1.0 mL dichloromethane). The reaction was stirred for 2 days and ¹H NMR showed formation of a mixture of ketone 17a and cyclic carbinolamine 18a in a 1:0.11 ratio, respectively, and this ratio remained unchanged over this time period.

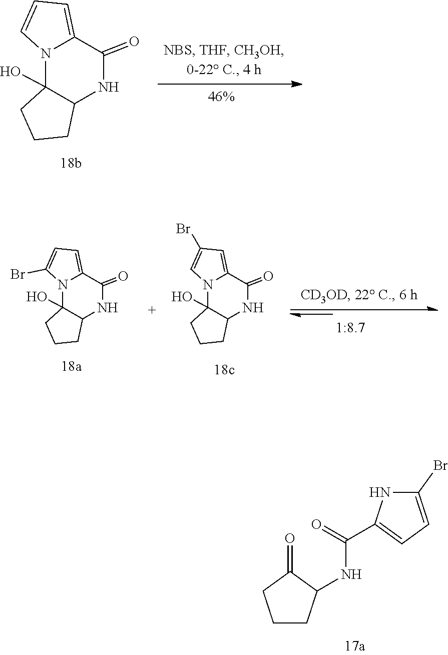 [SMILES: O=C1NC2CCCC2(O)n2cccc21 ; O=C1NC2CCCC2(O)n2c(Br)ccc21 ; O=C1NC2CCCC2(O)n2cc(Br)cc21 ; O=C(NC1CCCC1=O)c1ccc(Br)[nH]1]

In a 1.5 dram vial, carbinolamine 18b (2.6 mg, 0.014 mmol, 1.0 equiv) was dissolved in THF (0.14 mL) and MeOH (0.14 mL) and the mixture was cooled to 0° C. N-bromosuccinamide (2.5 mg, 0.0142 mmol, 1.05 equiv) was added and the reaction was allowed to warm up to ambient temperature (22° C.). After stirring for 4 h, TLC indicated there was no more starting material and the reaction mixture was concentrated and purified by column chromatography on silica (MeOH/CH₂Cl₂, 0 to 20%) to give a mixture of 18a/18c/17a (2.4 mg, 65%) in 1:0.6:0.44 ratio. This mixture was monitored by NMR to probe the stability of the 5-bromo derivative in CD₃OD. After 6 h, the mixture gave a ratio of 18a/18c/17a=0.15:0.6:1.30, and the ratio remained unchanged over 16 h.

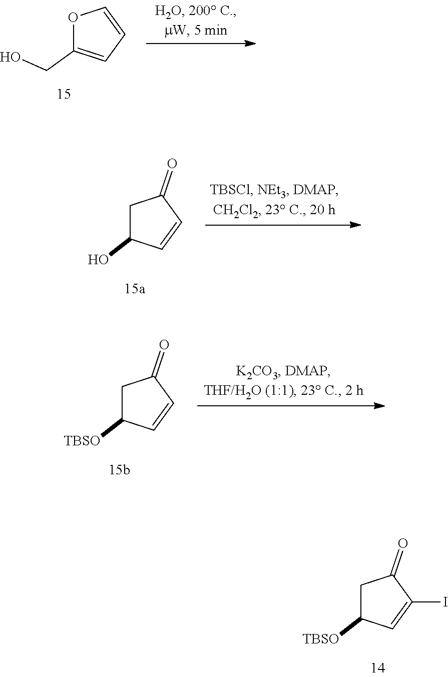 [SMILES: OCc1ccco1 ; O=C1C=C[C@@H](O)C1 ; CC(C)(C)[Si](C)(C)O[C@@H]1C=CC(=O)C1 ; CC(C)(C)[Si](C)(C)O[C@@H]1C=C(I)C(=O)C1]

Iodide 14 was prepared by following a described procedure ((a) Yang, P.; Yao, M.; Li, J.; Li, Y.; Li, A. Total Synthesis of Rubriflordilactone B. Angew. Chem. Int. Ed. 2016, 55, 6964-6968. (b) Saitman, A.; Theodorakis, E. A. Synthesis of a Highly Functionalized Core of Verrillin. Org. Lett. 2013, 15, 2410-2413. (c) Truax, N.J.; Ayinde, S.; Van, K.; Liu, J. O.; Romo, D. Pharmacophore-Directed Retrosynthesis Applied to Rameswaralide: Synthesis and Bioactivity of Sinularia Natural Product Tricyclic Cores. Org. Lett. 2019, 21, 7394-7399).

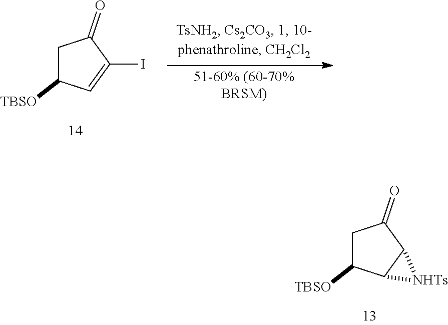

To a stirred solution of iodide 14 (1.0 g, 2.96 mmol, 1 equiv) in dichloromethane (anhydrous, 25 mL) in 50 mL round bottom flask was added 1, 10-phenathroline (598 mg, 3.32 mmol, 1.12 equiv) and p-toluenesulfonamide (1.01 g, 5.91 mmol, 2 equiv). The mixture was then cooled by ice bath, and cesium carbonate (982 mg, 3.02 mmol, 1.02 equiv) was added in 5 batches over 20 min to form a light yellow suspension. After stirring for 10 min, the ice bath was removed and the reaction was stirred at ambient temperature (22° C.) for 5 h. The reaction was quenched by adding water (30 mL), brine (20 mL), and extracted with dichloromethane (30 mL×3). The organic phase was combined, washed with brine (30 mL), dried over Na₂SO₄, and concentrated in vacuo. The residue was purified by automated column chromatography on silica (wet loaded, acetone/hexane, 0 to 40%) to give the aziridine 13 (0.68 g, 60%, 70% based on recovered starting material) as an off white solid and recovered iodide (0.14 g).

When the reaction was scaled-up to 8.8 g of iodoenone 14 in dichloromethane (200 mL) with 1, 10-phenathroline (5.24 g),p-toluenesulfonamide (8.89 g), and cesium carbonate (8.63 g), this provided aziridine 13 (5.05 g, 51%, 60% BRSM) along with recovered iodoenone 14 (1.32 g).

13: Off white solid. ¹H NMR (CDCl₃, 400 MHz) δ 7.80 (d, J=8.3 Hz, 2H), 7.36 (d, J=8.3 Hz, 2H), 4.55 (d, J=5.5 Hz, 1H), 3.71 (d, J=4.5 Hz, 1H), 3.30 (d, J=4.4 Hz, 1H), 2.50 (dd, J=18.3, 5.6 Hz, 1H), 2.45 (s, 3H), 1.93 (d, J=18.3 Hz, 1H), 0.87 (s, 9H), 0.11 (s, 3H), 0.07 (s, 3H); ¹³C NMR (100 MHz, CDCl₃) δ 205.2, 145.6, 134.2, 130.2 (2), 128.1 (2), 68.5, 48.1, 44.9, 43.5, 25.8 (3), 21.9, 18.1, −4.6, −4.7; IR (thin film, cm⁻¹) 3069, 2930, 2857, 1757, 1337, 1159, 1086; HRMS (ESI) calcd for C₁₈H₂₈N₇O₄SSi⁺ [M+Na]⁺404.1322, found 404.1327.

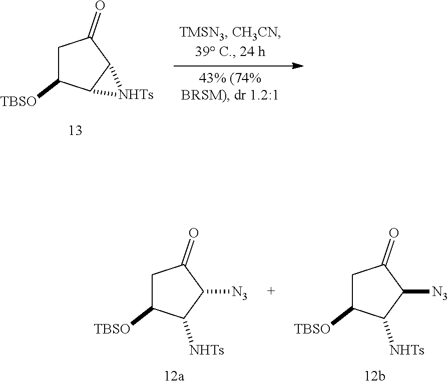

To a stirred solution of aziridine 13 (5.05 g, 13.2 mmol, 1 equiv) in a 350 mL pressure vessel (as a sealed flask) was added acetonitrile (anhydrous, 104 mL), and trimethylsilyl azide (2.08 mL, 15.8 mmol, 1.2 equiv). The flask was sealed and heated to 39° C. for 24 h. The brown mixture was then concentrated and purified by MPLC on silica using acetone/hexane (wet loaded, 0 to 50%) to give the desired diastereomer 12b (1.5 g, 27%, 47% BRSM) as a yellow solid, and the undesired diastereomer 12a (0.87 g, 16%, 27% BRSM) as a yellow solid along with recovered starting material 13 (2.25 g, 46%).

12a: Yellow solid. ¹H NMR (600 MHz, CDCl₃) δ 7.75 (d, J=8.2 Hz, 2H), 7.35 (d, J=8.1 Hz, 2H), 4.95 (d, J=3.0 Hz, 1H), 4.65 (app dt, J=5.5, 1.7 Hz, 1H), 4.23 (dd, J=6.4, 1.4 Hz, 1H), 3.57-3.40 (m, 1H), 2.69 (dd, J=19.1, 5.4 Hz, 1H), 2.44 (s, 3H), 2.42 (d, J=19.2 Hz, 1H), 2.22 (d, J=19.2 Hz, 1H), 0.84 (s, 9H), 0.09 (s, 3H), 0.07 (s, 3H); ¹³C NMR (150 MHz, CDCl₃) δ 210.1, 144.4, 135.7, 130.1, 127.4, 70.1, 62.8, 58.9, 42.7, 25.7, 21.7, 17.9, −4.8, −5.0; IR (thin film, cm-1): 2113, 1699, 1162, 833; HRMS (ESI) calcd for C₁₈H₂₈N₄NaO₄SSi⁺ [M+Na]⁺ 447.1493, found 447.1496.

12b: Yellow solid. ¹H NMR (600 MHz, CDCl₃) δ 7.80 (d, J=8.3 Hz, 2H), 7.33 (d, J=8.2 Hz, 2H), 5.53 (d, J=6.7 Hz, 1H), 4.25 (q, J=7.1 Hz, 1H), 3.76 (app d, J=8.9 Hz, 1H), 3.43 (dt, J=9.1, 6.8 Hz, 1H), 2.77 (ddd, J=18.8, 7.2, 1.6 Hz, 1H), 2.43 (s, 3H), 2.26 (dd, J=18.8, 7.3 Hz, 1H), 0.82 (s, 9H), 0.04 (s, 3H), 0.03 (s, 3H); ¹³C NMR (150 MHz, CDCl₃) δ 206.4, 144.2, 137.3, 130.0 (2), 127.3 (2), 70.5, 67.7, 63.7, 45.3, 25.7 (3), 21.7, 18.0, −4.72, −4.73; IR (thin film, cm-1): 3238, 2110, 1759, 1328, 1151; HRMS (ESI) calcd for C₁₈H₂₈N₄NaO₄SSi⁺ [M+Na]⁺ 447.1493, found 447.1496.

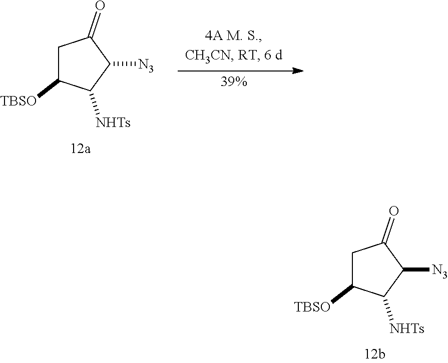

To a 100 mL round-bottom flask was added the azide 12a (3.45 g, 8.12 mmol) and acetonitrile (anhydrous, 68 mL), followed by the addition of 4A molecular sieves powder (10.35 g) at ambient temperature (22° C.), and the mixture was stirred for 6 days. NMR shows it formed a mixture of two diastereomers in ˜1:1 ratio. The mixture was filtered and washed with acetonitrile (50 m). The solvent concentrated in vacuo and flash column chromatography using acetone/hexane (wet loaded, 0 to 50%) gave the desired diastereomer 12b (1.35 g, 39% yield) and recovered 12a (1.15 g, 33% recovered).

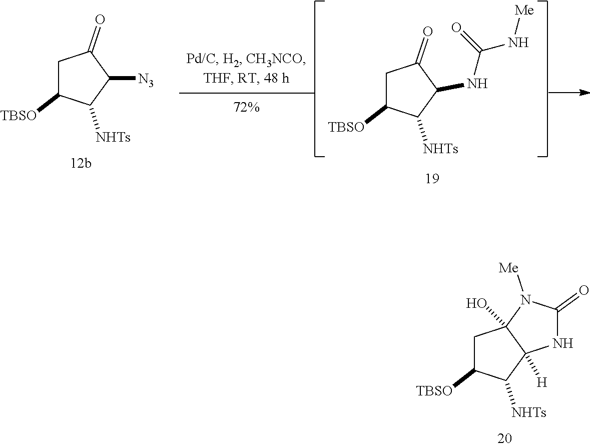

To a 250 mL round-bottom flask was charged 12b (2.19 g, 5.16 mmol, 1.0 equiv) and THF (103 mL) followed by 10% palladium on carbon (1.19 g, 2.58 mmol, 0.5 equiv) at ambient temperature (22° C.). A H₂ bladder (equipped with a valve) was connected and the flask was put under vacuum and backfilled with H₂ for 3 times. Methyl isocyanate (0.35 mL, 5.67 mmol, 1.1 equiv) was added and another H₂ balloon was connected. The reaction was stirred at ambient temperature for 48 h and TLC indicated there was no more starting material. The mixture was filtered through a cotton plug and washed with THF (10 mL) and concentrated in vacuo. The residue was purified by MPLC on silica (dry loaded, MeOH/CH₂Cl₂ 0 to 5%) to give 20 (1.7 g, 72% yield) as a light yellow solid.

20: Light yellow solid. ¹H NMR (600 MHz, CD₃OD) δ 7.79 (d, J=8.1 Hz, 2H), 7.39 (d, J=8.2 Hz, 2H), 4.23-4.01 (m, 1H), 3.59 (dd, J=2.9, 1.0 Hz, 1H), 3.13 (ddd, J=4.0, 2.8, 1.0 Hz, 1H), 2.71 (s, 3H), 2.43 (s, 3H), 2.16 (dd, J=13.8, 5.2 Hz, 1H), 1.99 (ddd, J=13.8, 4.5, 1.2 Hz, 1H), 0.77 (s, 9H), −0.05 (s, 3H), −0.10 (s, 3H); ¹³C NMR (150 MHz, CD₃OD) δ 160.7, 145.0, 138.9, 130.9 (2), 128.3 (2), 96.4, 77.0, 69.3, 69.0, 42.8, 26.2 (3), 24.6, 21.5, 18.7, −4.9, −5.0; IR (thin film, cm⁻¹) 3093, 1690, 1327, 1160, 1083; HRMS (ESI) calcd. for C₂₀H₃₃N₃NaO₅SSi⁺ [M+Na]⁺ 478.1802, found 478.1807.

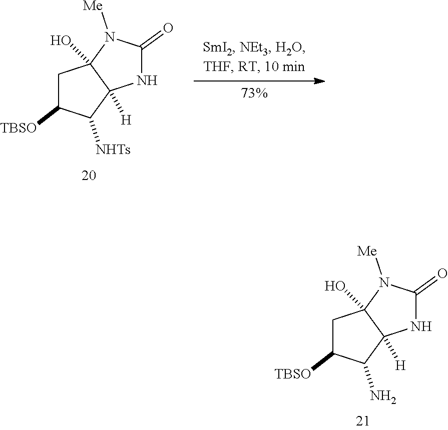

To a 500 mL round-bottom flask was charged 20 (0.83 g, 1.82 mmol, 1.0 equiv) and the flask was put under vacuum and backfilled with argon for 3 times. Degassed DI water (by freeze-pump-thaw method, 0.98 mL, 54.7 mmol, 30 equiv) and samarium iodide solution (in THF, 0.078 M, 234 mL, 18.2 mmol, 10 equiv) was sequentially added at ambient temperature (22° C.). Triethylamine (freshly distilled over CaH₂, 5.1 mL, 36.4 mmol, 20 equiv) was added dropwise over 10 min. TLC indicated the starting material was all consumed. The reaction mixture was filtered, and the liquid phase was collected, while the solid was transferred to six centrifuge tubes (10 mL/tube), and extracted with 10 mL CH₃OH—CH₂Cl₂ (10%, v/v) in each tube. The suspension was centrifuged, and the clear supernatant was collected, while solid was extracted again. The process was repeated 6-10 times until no more product was seen on TLC. The organic solution was combined and concentrated, and the residue was purified by MPLC on silica (with ELSD detector, dry loading method) with CH₃OH/CH₂Cl₂(0 to 10%) to give amine 21 (0.5 g, 73%) as a white powder.

21: White solid; ¹H NMR (600 MHz, CD₃OD) δ 4.02 (app q, J=6.5 Hz, 1H), 3.33 (d, J=5.5 Hz, 1H), 2.84 (app t, J=5.9 Hz, 1H), 2.75 (s, 3H), 2.27 (dd, J=13.4, 6.0 Hz, 1H), 1.91 (dd, J=13.3, 7.2 Hz, 1H), 0.90 (s, 9H), 0.11 (dd, J=11.0, 1.4 Hz, 6H); ¹³C NMR (150, MHz, CD₃OD) δ 161.0, 94.7, 77.2, 67.91, 67.85, 44.0, 26.3 (3), 24.5, 18.8, −4.6, −4.7; IR (thin film, cm⁻¹) 2920, 1660, 1467; HRMS (ESI) calcd. For C₁₃H₂₈N₃O₃Si⁺[M+H]⁺ 302.1894, found 302.1898.

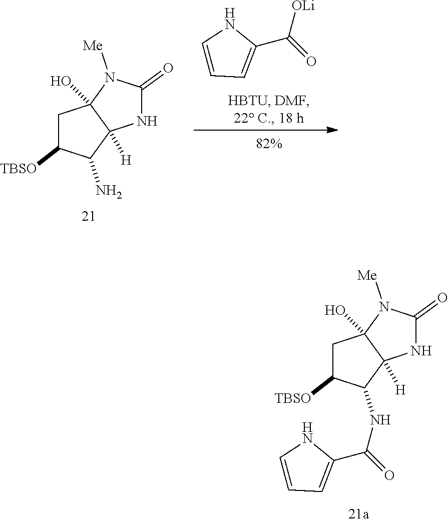

To a 100 mL round-bottom flask was charged pyrrole-2-carboxylic acid (191.6 mg, 1.73 mmol, 2.0 equiv), DI water (17 mL) followed by lithium hydroxide monohydrate (74.5 mg, 1.78 mmol, 2.06 equiv). The mixture was stirred for 15 min and the solids dissolved. The solution was then dried under high vacuum to get lithium pyrrole carboxylate as a white solid. To this solid was added DMF (20 mL) and hexafluorophosphate benzotriazole tetramethyl uronium (HBTU, 654.0 mg, 1.73 mmol, 2.0 equiv), and the mixture was stirred at ambient temperature (22° C.) for 1 h. In a separate 100 mL flask, amine (260 mg, 0.86 mmol, 1.0 equiv) and DMF (anhydrous, 60 mL) were added sequentially and the mixture was heated at ˜100° C. to became a clear solution. The mixture was cooled back to ambient temperature (22° C.) and was added via cannula to the flask containing acid pyrrole-2-carboxylic acid and HBTU. The mixture was stirred for 18 h until TLC analysis indicated complete consumption of amine 21. The reaction was put under high vacuum to remove the solvent, and the organic residue was purified by MPLC on silica (dry loading) using ethyl acetate/hexane (0 to 100%) followed by methanol/dichloromethane (0 to 10%) to give the amide 21a (278.8 mg, 82% yield) as a white solid which was carried on directly to the next step.

21a: White solid. ¹H NMR (600 MHz, CD₃OD) δ 6.92 (dd, J=2.5, 1.4 Hz, 1H), 6.83 (dd, J=3.7, 1.4 Hz, 1H), 6.17 (dd, J=3.7, 2.5 Hz, 1H), 4.70 (s, 1H), 4.37 (dt, J=7.3, 6.5 Hz, 1H, 1H), 4.00 (dd, J=7.1, 5.9 Hz, 1H), 3.64 (d, J=5.9 Hz, 1H), 2.78 (s, 3H), 2.31 (dd, J=13.4, 6.3 Hz, 1H), 1.99 (dd, J=13.4, 7.9 Hz, 1H), 0.85 (s, 9H), 0.08 (s, 3H), 0.04 (s, 3H); ¹³C NMR (150 MHz, CD₃OD) δ 163.7, 161.1, 126.8, 123.1, 111.9, 110.2, 94.0, 75.1, 66.8, 66.3, 44.3, 26.2, 24.4 (3), 18.8, −4.6, −4.8; IR (thin film, cm⁻¹): 3318, 1671, 1637, 1408, 1070; HRMS (ESI) calcd for C₁₈H₃₀N₄NaO₄Si⁺ [M+Na]⁺ 417.1929, found 417.1932.

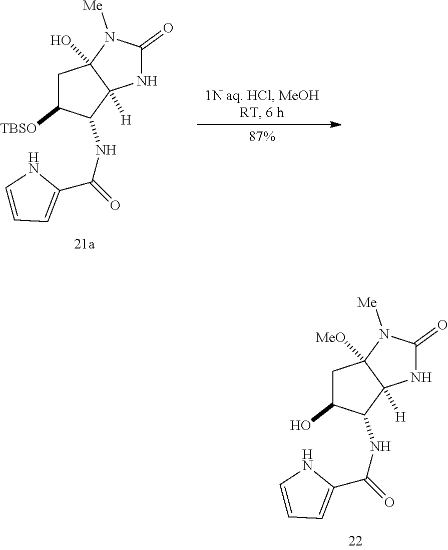 [SMILES: CN1C(=O)N[C@H]2[C@H](NC(=O)c3ccc[nH]3)[C@@H](O[Si](C)(C)C(C)(C)C)C[C@]21O ; CO[C@@]12C[C@H](O)[C@@H](NC(=O)c3ccc[nH]3)[C@@H]1NC(=O)N2C]

To a stirred solution of crude 21a (398 mg, 0.755 mmol, 1.0 equiv) was added methanol(18.0 mL), followed by 1N aq. hydrochloric acid (0.75 mL, 0.75 mmol, 1.0 equiv). It turned to a cloudy suspension but became clear as the reaction proceeded. After stirring for 6 h, TLC indicated complete consumption of starting material, the reaction was neutralized with a pH 7 buffer. The reaction was then concentrated and the residue was purified by MPLC on silica (dry loading, methanol/dichloromethane (0 to 10%) to give the alcohol 22 (193.4 mg, 87%) as a white solid.

22: White solid. ¹H NMR (500 MHz, CD₃OD) δ 6.92 (dd. J=2.6, 1.4 Hz, 1H), 6.86 (dd, J=3.8, 1.4 Hz, 1H), 6.17 (dd, J=3.7, 2.5 Hz, 1H), 4.22 (ddd, J=9.2, 8.2, 6.8 Hz, 1H), 3.91 (dd, J=8.3, 6.5 Hz, 1H), 3.74 (d, J=6.4 Hz, 1H), 3.07 (s, 3H), 2.73 (s, 3H), 2.36 (ddd, J=13.3, 6.8, 0.9 Hz, 1H), 2.00 (dd, J=13.4, 9.2 Hz, 1H); ¹³C NMR (125 MHz, CD₃OD)) δ 164.3, 161.7, 126.6, 123.1, 112.1, 110.2, 97.8, 72.9, 66.7, 61.5, 49.6, 43.5, 24.6; IR (thin film, cm⁻¹): 3290, 1679, 1625, 1405, 1064; HRMS (ESI) calcd for C₁₃H₁₈N₄NaO₄⁺[M+Na]⁺ 317.1220, found 317.1224.

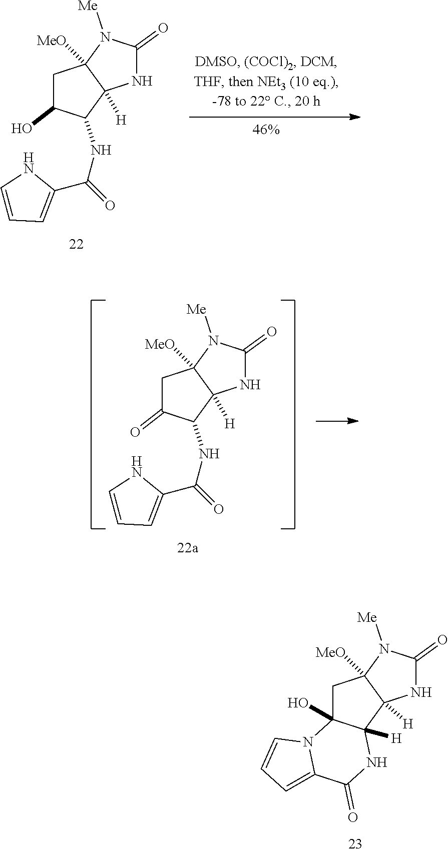

In a 2.0 dram vial, alcohol 22 (31.0 mg, 0.11 mmol, 1.0 equiv) was dissolved in THF (1.2 mL) and dimethylsulfoxide (anhydrous, 0.12 mL), and the mixture was cooled to −78° C. In a separate 1.0 dram vial, oxalyl chloride (21 uL, 0.24 mmol, 2.0 equiv) was dissolved in dichloromethane (0.6 mL), and the mixture was cooled to −78° C., and DMSO (21 uL, 0.03 mmol, 2.8 equiv) was added. After stirring for 30 min, the solution was transferred via cannula to the 2.0 dram vial containing 22 at −78° C. The reaction was stirred for 1 h, and freshly distilled triethylamine (0.15 mL, 1.07 mmol, 10 equiv) was added and the reaction was warmed up to −30° C. within 1 h. A second batch of triethylamine (0.15 mL, 1.07 mmol, 10 equiv) was added and the reaction was warmed up to ambient temperature (22° C.) and stirred for 18 h. The reaction was filtered through a cotton plug and washed with THF (1 mL), and the organic phase was combined and concentrated under high vacuum to remove DMSO. The residue was purified by column chromatography on silica (dry loading) using methanol/dichloromethane (0 to 10%) to give carbinolamine 23 (14.2 mg, 46%) as an off white solid.

23: Off white solid. ¹H NMR (500 MHz, CD₃OD) δ 7.22 (dd, J=2.8, 1.6 Hz, 1H), 6.93 (dd, J=3.8, 1.6 Hz, 1H), 6.30 (dd, J=3.8, 2.7 Hz, 1H), 3.80 (d, J=4.4 Hz, 1H), 3.74 (d, J=4.5 Hz, 1H), 2.98 (s, 3H), 2.76 (s, 3H), 2.75 (d, J=13.5 Hz, 3H), 2.63 (d, J=14.4 Hz, 1H); ¹³C NMR (125 MHz, CD₃OD) δ 161.9, 161.3, 123.3, 123.2, 116.2, 111.7, 98.0, 87.4, 69.1, 62.9, 49.8, 46.8, 24.8; IR (cm¹, thin film): 3251, 1687, 1646, 1557, 1459, 1315, 1072; HRMS (ESI): calcd for C₁₃H₁₆N₄NaO₄⁺ [M+Na]⁺ 315.1064, found 315.1066.

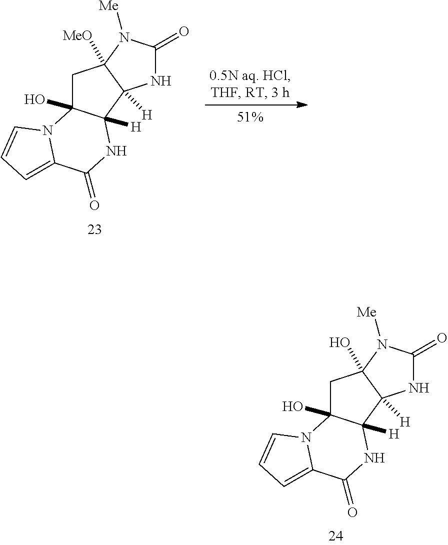 [SMILES: CO[C@@]12C[C@]3(O)[C@@H](NC(=O)c4cccn43)[C@@H]1NC(=O)N2C ; CN1C(=O)N[C@H]2[C@@H]3NC(=O)c4cccn4[C@]3(O)C[C@]21O]

To a 1.5 dram vial was charged aminal 23 (6.8 mg, 0.023 mmol, 1.0 equiv), THF (1.5 mL) and 0.5 N aqueous hydrochloric acid solution (23 uL, 0.012 mmol, 0.5 equiv). The reaction was stirred at ambient temperature (22° C.) for 3 h. The reaction was then adjusted to pH 7 with pH 7 buffer and concentrated in vacuo. The residue was purified by column chromatography on silica (dry loading) using methanol/dichloromethane (0 to 10%) to give hemiaminal 24 (3.3 mg, 51%, 65% based on recovered starting material) as a white solid, and recovered 23 (1.5 mg, 22%).

24: White solid. ¹H NMR (600 MHz, CD₃OD) δ 7.22 (dd, J=2.7, 1.6 Hz, 1H), 6.93 (dd, J=3.8, 1.6 Hz, 1H), 6.31 (dd, J=3.8, 2.7 Hz, 1H), 3.77 (app d, J=4.4 Hz, 1H), 3.59 (d, J=4.4 Hz, 1H), 2.80 (s, 3H), 2.73 (d, J=14.3 Hz, 1H), 2.60 (d, J=14.4 Hz, 1H); ¹³C NMR (150 MHz, CD₃OD) δ 161.4, 161.2, 123.23, 123.19, 116.1, 111.7, 93.7, 88.0, 69.2, 69.0, 47.4, 24.5; IR (thin film, cm⁻¹): 3305, 1679, 1643, 1557, 1462; HRMS (ESI): calcd for C₁₂H₁₄N₄NaO₄⁺ [M+Na]⁺ 301.0907, found 301.0910.

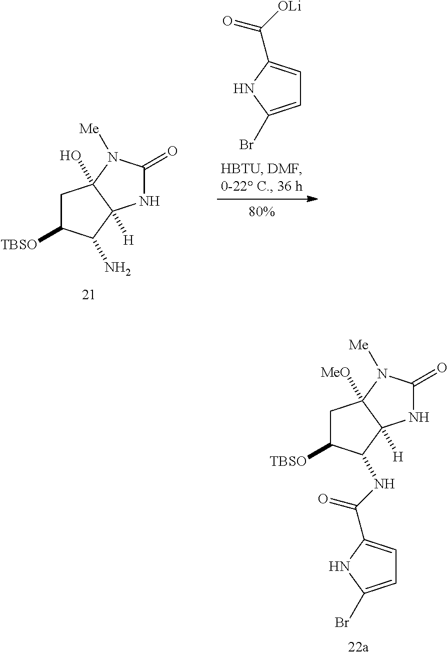

Following an identical procedure as described for synthesis of amide 22, 5-bromopyrrole-2-carboxylic acid (109.9 mg, 0.58 mmol, 3.8 equiv), DI water (3 mL), lithium hydroxide monohydrate (25.0 mg, 0.59 mmol, 3.92 equiv), hexafluorophosphate benzotriazole tetramethyl uronium (HBTU, 219.4 mg, 0.58 mmol, 3.8 equiv), and DMF (16.6 mL) were used, and the reaction was stirred for 36 h. Column chromatography on silica using methanol/dichloromethane (0 to 10%) to give 22a as a white solid (57.7 mg, 80%).

22a: White solid. ¹H NMR (600 MHz, CD₃OD) δ 6.77 (app d, J=3.8 Hz, 1H), 6.14 (app d, J=3.9 Hz, 1H), 4.29 (q, J=6.8 Hz, 1H), 3.97 (t, J=6.2 Hz, 1H), 3.80 (d, J=5.6 Hz, 1H), 3.08 (s, 3H), 2.73 (s, 3H), 2.29 (dd, J=13.6, 6.2 Hz, 1H), 2.03 (dd, J=13.6, 7.3 Hz, 1H), 0.85 (d, J=1.4 Hz, 9H), 0.07 (s, 3H), 0.05 (s, 3H); ¹³C NMR (150 MHz, CD₃OD) δ 162.5, 161.7, 128.6, 113.4, 112.5, 104.6, 98.6, 74.8, 66.4, 60.7, 49.7, 43.7, 26.2 (3), 24.7, 18.8, −4.6, −4.8; IR (thin film, cm⁻¹) 3271, 1687, 1632, 1396, 1102, 1082; HRMS (ESI): calcd for C₁₉H₃₀BrN₄O₄Si- [M−H]⁻ 485.1225, found 485.1246.

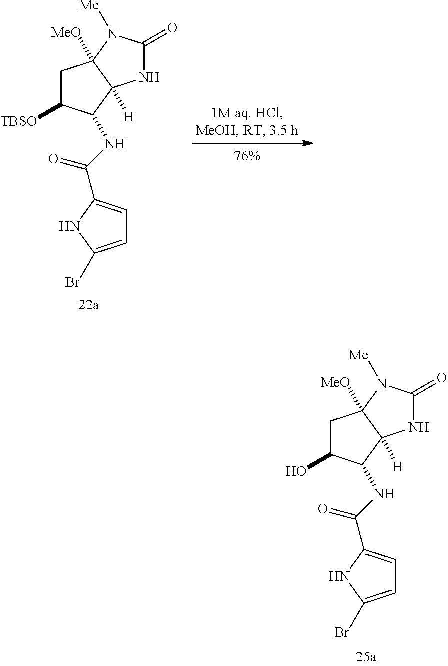 [SMILES: CO[C@@]12C[C@H](O[Si](C)(C)C(C)(C)C)[C@@H](NC(=O)c3ccc(Br)[nH]3)[C@@H]1NC(=O)N2C ; CO[C@@]12C[C@H](O)[C@@H](NC(=O)c3ccc(Br)[nH]3)[C@@H]1NC(=O)N2C]

To a stirred solution of 22a (12.5 mg, 0.026 mmol, 1.0 equiv) in methanol (0.25 mL) was added hydrochloric acid (1N aqueous solution, 31 ul, 0.031 mmol, 1.2 equiv) at ambient temperature. The reaction was stirred for 3.5 h and there was no starting material as indicated by TLC. The reaction was neutralized by pH 7 buffer to pH 7, and the solution was concentrated under high vacuum. The residue was purified by MPLC on silica (dry loading, MeOH/CH₂Cl₂ to 15%) to give alcohol 25a (7.3 mg, 76% yield) as a white solid.

25a: White solid. ¹H NMR (500 MHz, CD₃OD) δ 6.81 (d, J=3.9 Hz, 1H), 6.14 (d, J=3.9 Hz, 1H), 4.20 (td, J=8.9, 7.0 Hz, 1H), 3.89 (dd, J=8.3, 6.5 Hz, 1H), 3.72 (d, J=6.5 Hz, 1H), 3.07 (s, 3H), 2.73 (s, 3H), 2.36 (dd, J=13.4, 6.7 Hz, 1H), 1.99 (dd, J=13.4, 9.3 Hz, 1H); IR (thin film, cm⁻¹) 3311, 1670, 1638, 1529, 1066; HRMS (ESI) calcd for C₁₃H₁₇⁷⁹BrN₄NaO₄⁺ [M+Na]⁺ 395.0325, found 395.0327.

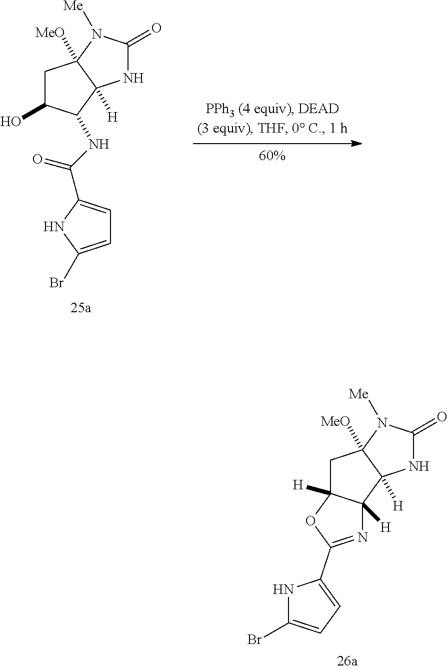 [SMILES: CO[C@@]12C[C@H](O)[C@@H](NC(=O)c3ccc(Br)[nH]3)[C@@H]1NC(=O)N2C ; CO[C@@]12C[C@H]3OC(c4ccc(Br)[nH]4)=N[C@H]3[C@@H]1NC(=O)N2C]

To a 1.5 dram vial was charged 25a (7.0 mg, 0.019 mmol, 1.0 equiv), triphenyl phosphine (20.0 mg, 0.076 mmol, 4.0 equiv) and THF (0.5 mL). The mixture was stirred for 5 min and cooled to 0° C. A solution of diethyl azodicarboxylate (9 uL, 0.057 mmol, 3.0 equiv) in THF (0.03 mL) was added. After stirring for 1 h, starting material 25a was all consumed as shown on TLC. The reaction mixture was concentrated in vacuo and the organic residue was purified by column chromatography on silica (dry loading, methanol/dichloromethane, 0 to 10%) to give oxazoline 26a (4.0 mg, 60%) as a white solid.

26a: White solid. ¹H NMR (600 MHz, CD₃OD) δ 6.74 (d, J=3.8 Hz, 1H), 6.18 (d, J=3.8 Hz, 1H), 5.13 (td, J=7.8, 5.4 Hz, 1H), 4.39 (d, J=7.9 Hz, 1H), 3.99 (d, J=1.7 Hz, 1H), 3.12 (s, 3H), 2.86-2.49 (m, 4H), 2.19 (dd, J=15.1, 5.5 Hz, 1H); ¹³C NMR (150 MHz, CD₃OD) δ 161.9, 159.4, 121.8, 116.2, 113.1, 105.1, 102.1, 83.1, 79.9, 63.4, 51.1, 42.4, 24.7; IR (thin film, cm-1) 1683, 1648, 1434, 1393, 1075, 1019; HRMS (ESI) cald for C₁₃H₁₆⁷⁹BrN₅O₃⁺ [M+H]⁺ 355.0400, found 355.0410.

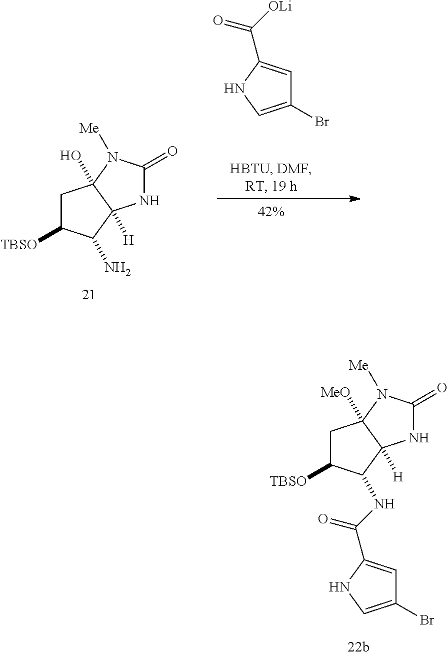

Following the procedure of making 22, 4-bromo pyrrole-2-carboxylic acid (16.6 mg, 0.087 mmol, 3.6 equiv), DI water (0.4 mL), lithium hydroxide monohydrate (3.7 mg, 0.087 mmol, 3.6 equiv), hexafluorophosphate benzotriazole tetramethyl uronium (HBTU, 34 mg, 0.087 mmol, 3.6 equiv), amine 21 (7.3 mg, 0.024 mmol, 1.0 equiv) and DMF (0.7 mL) were used, and the reaction was stirred for 18 h. Column chromatography on silica using CH₂Cl₂/MeOH (0 to 10%) gave 22b as a white solid (5.0 mg, 42% yield).

22b: White solid. ¹H NMR (400 MHz, CD₃OD) δ 6.92 (d, J=1.6 Hz, 1H), 6.83 (d, J=1.6 Hz, 1H), 4.59 (s, 1H), 4.36 (q, J=7.2 Hz, 1H), 4.02-3.93 (m, 1H), 3.63 (d, J=5.9 Hz, 1H), 2.77 (s, 3H), 2.30 (dd, J=13.3, 6.3 Hz, 1H), 1.98 (dd, J=13.4, 8.0 Hz, 1H), 0.85 (s, 9H), 0.06 (d, J=11.2 Hz, 6H).

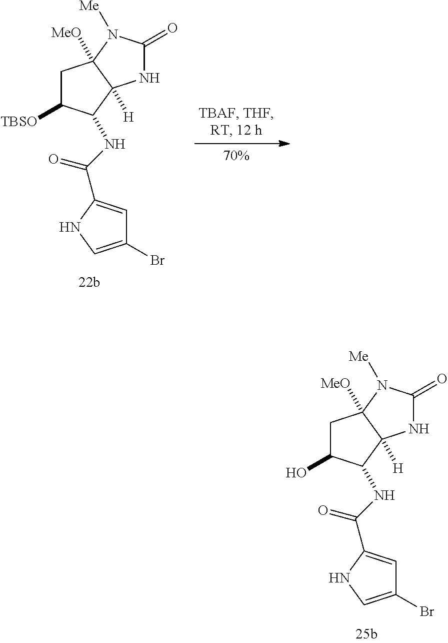 [SMILES: CO[C@@]12C[C@H](O[Si](C)(C)C(C)(C)C)[C@@H](NC(=O)c3cc(Br)c[nH]3)[C@@H]1NC(=O)N2C ; CO[C@@]12C[C@H](O)[C@@H](NC(=O)c3cc(Br)c[nH]3)[C@@H]1NC(=O)N2C]

To a stirred solution of 22b (28 mg, 0.057 mmol) in THF (1.0 mL) was added TBAF (1M solution in THF, 0.11 mL, 0.11 mmol, 2.0 equiv) at ambient temperature (22° C.). The mixture was stirred for 12 h and starting material was all consumed as indicated on TLC. The mixture was concentrated under reduced pressure and the residue was purified by column chromatography on silica using MeOH/CH₂Cl₂(0 to 10%) to give 25b (15.0 mg, 70% yield) as a colorless oil.

25b: Colorless oil. ¹H NMR (600 MHz, CD₃OD) δ 6.93 (d, J=1.6 Hz, 1H), 6.87 (d, J=1.6 Hz, 1H), 4.20 (td, J=9.0, 6.9 Hz, 1H), 3.89 (dd, J=8.4, 6.6 Hz, 1H), 3.73 (d, J=6.5 Hz, 1H), 3.07 (s, 3H), 2.73 (s, 3H), 2.36 (dd, J=13.4, 6.8 Hz, 1H), 1.99 (dd, J=13.4, 9.3 Hz, 1H).

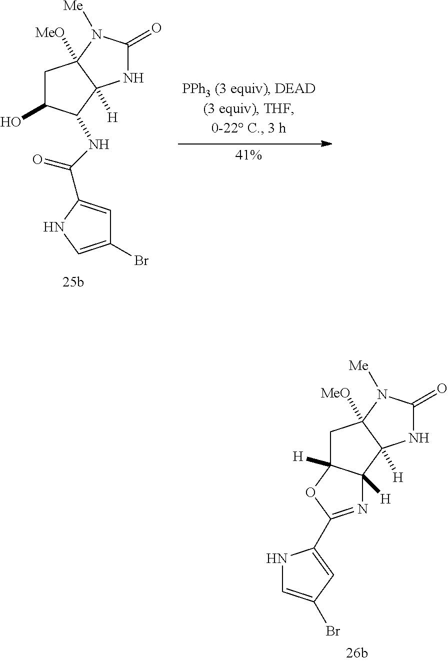 [SMILES: CO[C@@]12C[C@H](O)[C@@H](NC(=O)c3cc(Br)c[nH]3)[C@@H]1NC(=O)N2C ; CO[C@@]12C[C@H]3OC(c4cc(Br)c[nH]4)=N[C@H]3[C@@H]1NC(=O)N2C]

To a 1.0 dram vial was charged alcohol 25b (2.8 mg, 0.0075 mmol, 1.0 equiv), triphenylphosphine (5.9 mg, 0.0225 mmol, 3.0 equiv), and THF (0.2 mL). The mixture was cooled to 0° C. and a stock solution of diethyl azodicarboxylate (DEAD, 10 ul, 0.0225 mmol, 3.0 equiv) was added (the stock solution of DEAD was made by dissolving 35 ul of DEAD in THF 0.1 mL). Another two batches of DEAD solution (10 ul) was added after every hour, and 25b was all consumed as indicated by TLC. The reaction was concentrated and the residue was purified by column chromatography on silica (dry loading, methanol/dichloromethane 0 to 10%). The product was further purified by semi-prep HPLC to give oxazoline 26b (1.1 mg, 41%) as a colorless oil for characterization.

26b: Colorless oil. ¹H NMR (600 MHz, CD₃OD) δ 6.96 (d, J=1.7 Hz, 1H), 6.76 (d, J=1.7 Hz, 1H), 5.15 (td, J=7.8, 5.4 Hz, 1H), 4.41 (dd, J=8.3, 1.8 Hz, 1H), 3.99 (d, J=1.8 Hz, 1H), 3.12 (s, 3H), 2.75 (dd, J=15.1, 7.5 Hz, 1H), 2.70 (s, 3H), 2.19 (dd, J=15.1, 5.4 Hz, 1H); ¹³C NMR (150 MHz, CD₃OD) δ 161.9, 159.3, 123.7, 121.1, 116.1, 102.1, 98.0, 83.3, 79.9, 63.4, 51.1, 42.4, 24.7.

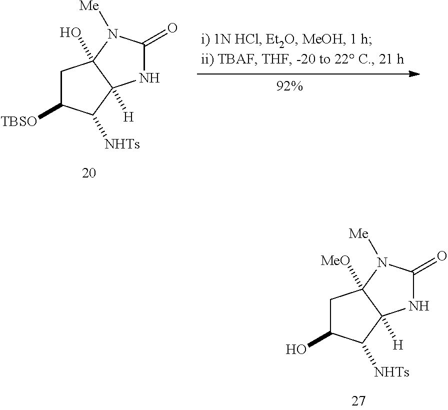

To a 100 mL round-bottom flask was charged the silyl ether 20 (1.40 g, 3.07 mmol, 1.0 equiv) and methanol (61 mL). The mixture was cooled to 0° C. and 1M hydrochloric acid in diethyl ether solution (0.31 mL, 0.31 mmol, 0.1 equiv) was added dropwise. The solution was warmed to ambient temperature (22° C.) in 1 h and white solid formed. THF (10 mL) was added to dissolve the solid. After stirring for 0.5 h, the starting material was all reacted as indicated by TLC, and the reaction was concentrated in vacuo to remove all the solvents. The resulting solid was redissolved in THF (61 mL), and the solution was cooled to −20° C., and tetrabutylammonium fluoride (TBAF) solution (1M in THF, 3.38 mL, 3.38 mmol, 1.1 equiv) was added and the reaction was warmed up to ambient temperature (22° C.), and stirred for 21 h. The reaction was complete as indicated by TLC, and was concentrated in vacuo. The residue was purified by MPLC on silica (wet loaded, methanol/dichloromethane 0 to 10%) to give the alcohol 27 (1.0 g, 92%) as a white solid.

27: White solid. ¹H NMR (600 MHz, CD₃OD) δ 7.81 (d, J=8.0 Hz, 2H), 7.38 (d, J=8.0 Hz, 2H), 3.98 (q, J=6.9 Hz, 1H), 3.70 (d, J=5.0 Hz, 1H), 3.14-2.92 (m, 4H), 2.66 (s, 3H), 2.43 (s, 3H), 2.22 (dd, J=13.6, 6.3 Hz, 1H), 1.89 (dd, J=13.6, 7.8 Hz, 1H); ¹³C NMR (150 MHz, CD₃OD) 161.5, 144.9, 138.7, 130.8 (2), 128.4 (2), 98.8, 73.8, 69.0, 62.4, 49.70, 42.6, 24.7, 21.5.; IR (thin film, cm⁻¹) 3393, 3147, 1703, 1159; HRMS (ESI) calcd for C₁₅H₂₁N₃NaO₅S⁺ [M+Na]⁺ 378.1094, found 378.1099.

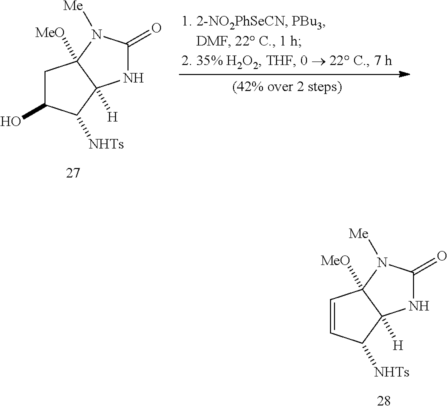

In a 25 mL round-bottom flask, alcohol 27 (210 mg, 0.59 mmol, 1.0 equiv) was dissolved in anhydrous N,N-dimethylformaldehyde (5.9 mL). The reaction flask was kept at ambient temperature in a water bath, and 2-nitrophenyl selenocyanate (536.7 mg, 2.36 mmol, 4.0 equiv) was added and the mixture became a clear red solution. To this solution was added tributylphosphine (0.59 mL, 2.36 mmol, 4.0 equiv) dropwise and the mixture was kept for 1 h, and 27 was all consumed as indicated by TLC. The mixture was then cooled to 0° C., and methanol (11.8 mL) was added. After stirring for 10 min at ambient temperature (22° C.), the mixture was concentrated under high vacuum to remove solvents, and the residue was purified by MPLC on silica (dry loading, CH₃OH/CH₂Cl₂, 0 to 5%) to give a mixture of the selenide intermediate and tributylphosphine oxide (0.88 g). This intermediate was subsequently dissolved in THF (4.62 mL) at 0° C., and hydrogen peroxide (35%, 0.12 mL, 1.58 mmol) was added dropwise. After warming up to ambient temperature (22° C.) over 1 h, the mixture was stirred for another 6 h. Solid of sodium sulfite (1.0 g) was added, and the reaction was stirred for 5 min and concentrated in vacuo. The residue was purified by MPLC on silica (dry loading, MeOH/CH₂Cl₂, 0 to 10%) to give a mixture (284 mg) of alkene 28 and POBu₃ (containing the desired product ca. 83.8 mg, 42% over 2 steps). The product was repurified by MPLC (dry loading, acetone/dichloromethane, 0 to 50%) to give pure 28 as a light yellow oil for characterization purpose.

28: Light yellow oil. ¹H NMR (400 MHz, CDCl₃) δ 7.77 (d, J=8.2 Hz, 2H), 7.34 (d, J=8.0 Hz, 2H), 6.04 (dd, J=5.9, 2.2 Hz, 1H), 5.66 (dd, J=5.9, 2.2 Hz, 1H), 5.14 (s, 1H), 5.03 (d, J=8.6 Hz, 1H), 4.03 (dq, J=8.5, 2.3 Hz, 1H), 3.88 (q, J=2.0 Hz, 1H), 3.15 (s, 3H), 2.72 (s, 3H), 2.44 (s, 3H); ¹³C NMR (100 MHz, CDCl₃) δ 158.6, 144.4, 136.9, 133.3, 133.0, 130.2 (2), 127.3 (2), 102.8, 66.7, 63.9, 50.1, 25.2, 21.7; IR (thin film, cm-1) 3374, 1694, 1447, 1331, 1160; HRMS (ESI) calcd for C₁₅H₁₉N₃NaO₄S⁺ [M+Na]⁺360.0988, found 360.0991.

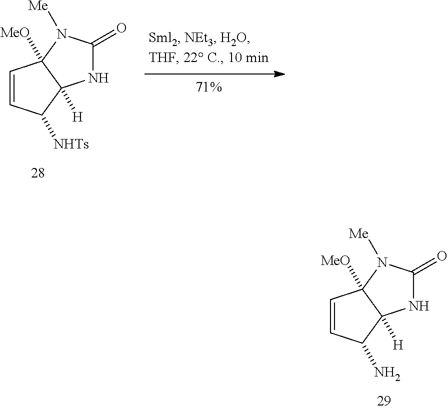

To a 100 mL round bottom flask was charged 28 (0.8 g mixture containing 0.24 g POBu, 0.56 g, 1.66 mmol, 1.0 equiv) and the flask was put under vacuum and backfilled with argon for 3 times. Degassed water (by freeze-pump-thaw method, 0.6 mL, 33.2 mmol, 20 equiv) and samarium iodide solution (in THF, 0.1 M, 166 mL, 16.6 mmol, 10 equiv) was sequentially added at ambient temperature (22° C.). Triethylamine (freshly distilled over CaH₂, 6.9 mL, 49.8 mmol, 30 equiv) was added dropwise over 10 min. TLC indicated the starting material was all consumed. The reaction was filtered, and the mother liquor was collected, while the solid was collected and transferred to six 10 mL centrifuge tubes, and extracted with 10 mL CH₃OH—CH₂Cl₂(20%, v/v, containing 1% NEt₃) in each tube. This process was repeated 6-10 times until no more product was seen on TLC. The liquid was collected and concentrated, and the residue was purified by MPLC on silica (equipped with ELSD detector, dry loading, CH₃OH/CH₂Cl₂ 0 to 10% containing 1% NEt₃) to give the amine 29 (214.4 mg, 71%) as a colorless oil.

29: Colorless oil. ¹H NMR (400 MHz, CD₃OD) δ 6.04 (dd, J=6.4, 1.4 Hz, 1H), 6.00 (dd, J=6.0, 2.0 Hz, 1H), 3.73 (d, J=2.1 Hz, 1H), 3.68 (d, J=2.2 Hz, 1H), 3.18 (s, 3H), 2.72 (s, 3H); ¹³C NMR (100 MHz, CD₃OD) δ 161.4, 138.9, 131.3, 105.2, 66.3, 64.9, 50.2, 25.3; IR (thin film, cm⁻¹) 3280, 2360, 2341, 1698; HRMS (ESI) calcd. for C₈H₁₃N₃NaO₂⁺ [M+Na]⁺ 206.0900, found 206.0900.

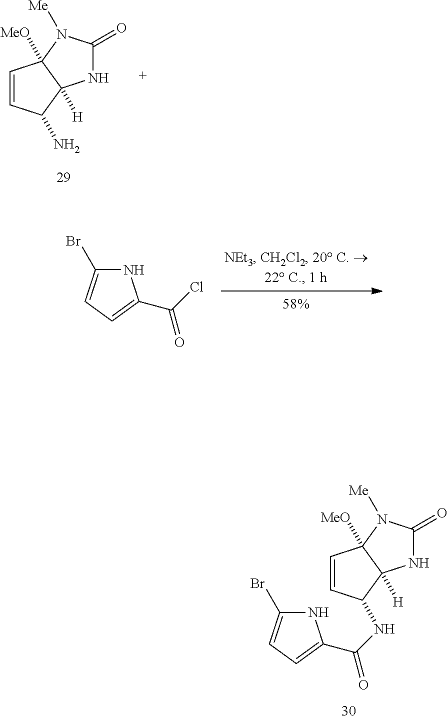 [SMILES: CO[C@@]12C=C[C@@H](N)[C@@H]1NC(=O)N2C ; O=C(Cl)c1ccc(Br)[nH]1 ; CO[C@@]12C=C[C@@H](NC(=O)c3ccc(Br)[nH]3)[C@@H]1NC(=O)N2C]

In a 1.0 dram vial, amine 29 (24.2 mg, 0.13 mmol, 1.0 equiv) was dissolved in dichloromethane (0.6 mL), and triethylamine (55 ul, 0.4 mmol, 3.0 equiv) was added and the mixture was cooled to −20° C. In a separate 1.0 dram vial, 2-bromo-1H-pyrole (37.6 mg, 0.2 mmol, 1.5 equiv) was dissolved in dichloromethane (0.7 mL) at 15° C., and N, N-dimethylformaldehyde (0.5 drop) was added, followed by the addition of oxalyl chloride (25 μl, 0.30 mmol, 2.25 equiv). The mixture was stirred for 30 min and cannulated to the other vial containing substrate 29 and triethylamine at −20° C. The mixture turned to be a cloudy light-yellow solution. After stirring for 15 min, the reaction was warmed up to ambient temperature. Methanol (1.0 mL) was added and stirred for 20 min. The reaction was concentrated in vacuo and the residue was purified by MPLC on silica (dry loading) using methanol/dichloromethane from 0 to 5%) to give the amide 30 as a white solid (27 mg, 58%).

30: White solid. ¹H NMR (500 MHz, CD₃SO₂CD₃) δ 12.23 (s, 1H), 8.34 (d, J=7.4 Hz, 1H), 7.19 (d, J=2.0 Hz, 1H), 6.84 (dd, J=3.8, 2.2 Hz, 1H), 6.22-6.08 (m, 2H), 5.95 (dd, J=5.9, 2.1 Hz, 1H), 4.61 (tt, J=4.6, 2.3 Hz, 1H), 3.78 (t, J=2.3 Hz, 1H), 3.06 (s, 3H), 2.62 (s, 3H); ¹³C NMR (125 MHz, CD₃SO₂CD₃) δ 159.1, 158.4, 135.2, 131.3, 127.6, 112.4, 110.9, 102.6, 102.3, 62.6, 61.8, 49.1, 24.7; IR (thin film, cm⁻¹) 3325, 2258, 1691, 1628, 1440, 1056; HRMS (ESI) calcd. for C13H15BrN4NaO3+[M+Na]+377.0220, found 377.0222.

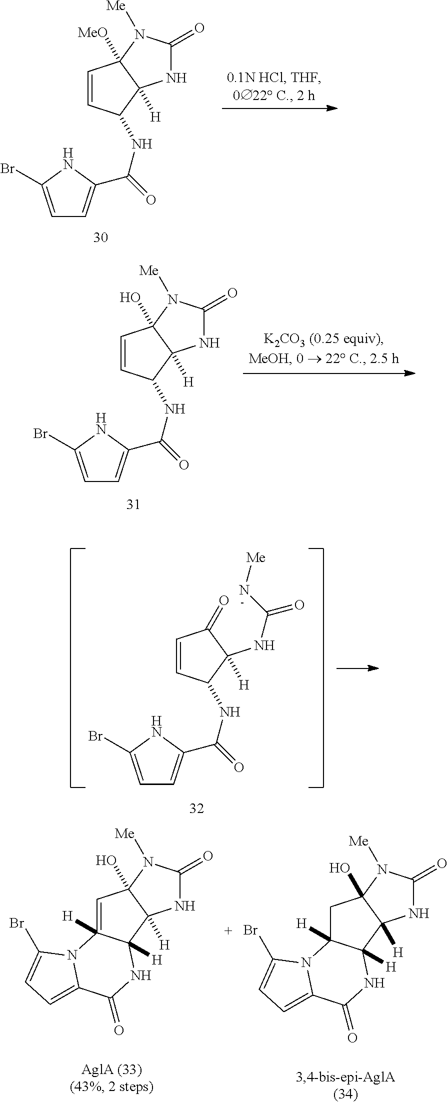

In a 10 mL round bottom flask, to a stirred solution of 30 (14.7 mg, 0.041 mmol, 1.0 equiv) in THF (4.1 mL) at 0° C. was added aqueous hydrochloride solution (0.1 M, 0.2 mL, 0.02 mmol, 0.5 equiv). The reaction was stirred for 10 min and warmed up to ambient temperature (22° C.) in 30 min. After stirring for 2 h, reaction was cooled back to 0° C. and sat. NaHCO₃ solution was added to adjust the pH to 7, and solvents were removed under high vacuum to give the crude intermediate 31 as a light yellow solid.

The crude 31 was dissolved in anhydrous methanol (1.3 mL), and the mixture was cooled to 0° C. Saturated K₂CO₃/MeOH solution (45 uL, 0.01 mmol, 0.25 equiv) was added and the reaction was warmed up to ambient temperature (22° C.) in 1 h. The solid was then filtered and washed with methanol (0.5 mL×2). The mother liquor was combined, concentrated and the residue was purified by column chromatography on silica (dry loading, methanol/dichloromethane 0 to 10%) to give agelastatin A as an off-white solid (1.7 mg). The solid cake from the reaction was dried and re-dissolved in DMSO (1.0 mL) and filtered through a cotton plug. DMSO was removed under high vacuum to give agelastatin A (4.3 mg) as an off-white solid in pure form (combined yield: 43%).

33: Off white solid. ¹H NMR (600 MHz, CD₃OD) δ 6.91 (d, J=4.1 Hz, 1H), 6.33 (d, J=4.1 Hz, 1H), 4.60 (app dt, J=12.1, 6.1 Hz, 1H), 4.09 (d, J=5.5 Hz, 1H), 3.88 (s, 1H), 2.81 (s, 3H), 2.65 (dd, J=13.1, 6.5 Hz, 1H), 2.10 (t, J=12.6 Hz, 1H); ¹³C NMR (150 MHz, CD₃OD) δ 161.4, 161.1, 124.1, 116.0, 113.8, 107.2, 95.7, 67.4, 62.2, 57.5, 54.4, 40.0, 24.2. Data matched previously reported.

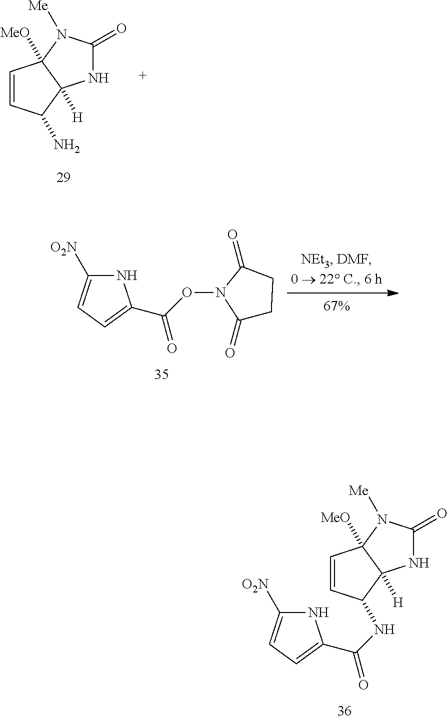 [SMILES: CO[C@@]12C=C[C@@H](N)[C@@H]1NC(=O)N2C ; O=C(ON1C(=O)CCC1=O)c1ccc([N+](=O)[O-])[nH]1 ; CO[C@@]12C=C[C@@H](NC(=O)c3ccc([N+](=O)[O-])[nH]3)[C@@H]1NC(=O)N2C]

To a 10 mL round-bottom flask was charged the amine 29 (53.0 mg, 0.29 mmol, 1 equiv) and DMF (anhydrous, 2.9 mL). The solution was cooled to 0° C. and triethyl amine (anhydrous, 0.20 mL, 1.43 mmol, 5.0 equiv) was added. The solid of 35 (87.9 mg, 0.35 mmol, 1.2 equiv) was added in one batch and the mixture was allowed to warm up to ambient temperature (22° C.) in 1 h. After stirring for another 5 h, the starting material was all consumed indicated by TLC. To the reaction mixture was added CH₃OH (1.0 mL), and solid precipitated. The solid was collected by filtration and washed with CH₃OH (0.2 mL) to give the product 36 (62.0 mg, 67%) as a light yellow solid. The product is of sufficient purity for the next steps.

36: Light yellow solid. ¹H NMR (500 MHz, CD₃SOCD₃) δ 12.77 (s, 1H), 8.76 (d, J=7.5 Hz, 1H), 7.92 (d, J=1.7 Hz, 1H), 7.55 (d, J=1.7 Hz, 1H), 7.23 (d, J=1.9 Hz, 1H), 6.18 (dd, J=5.9, 2.0 Hz, 1H), 5.96 (dd, J=5.9, 2.1 Hz, 1H), 4.63 (dq, J=7.1, 2.3 Hz, 1H), 3.82 (t, J=2.4 Hz, 1H), 3.07 (s, 3H), 2.62 (s, 3H); ¹³C NMR (125 MHz, CD₃SOCD₃) δ 158.9, 158.4, 136.3, 134.8, 131.6, 126.5, 122.9, 105.8, 102.4, 62.7, 61.5, 49.1, 24.7; IR (thin film, cm⁻¹) 1677, 1638, 1331, 1045; HRMS (ESI) calcd for C₁₃H₁₅N₅NaO₅⁺ [M+Na]⁺344.0965, found 344.0966.

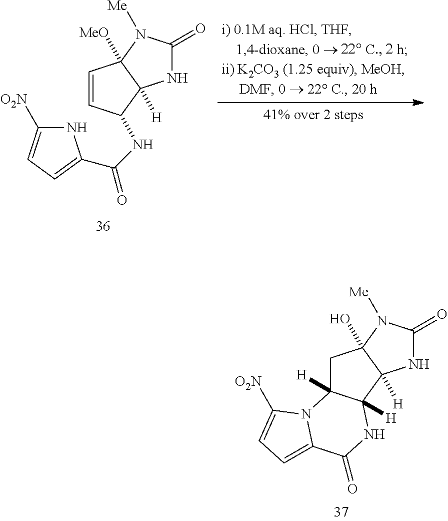 [SMILES: CO[C@@]12C=C[C@@H](NC(=O)c3ccc([N+](=O)[O-])[nH]3)[C@@H]1NC(=O)N2C ; CN1C(=O)N[C@H]2[C@@H]3NC(=O)c4ccc([N+](=O)[O-])n4[C@@H]3C[C@]21O]

To a stirred suspension of 36 (10.6 mg, 0.033 mmol, 1.0 equiv) in THF (1.5 mL) and 1, 3-dioxane (1.8 mL) at 0° C. was added aqueous HCl solution (0.1 M, 0.17 mL, 0.017 mmol, 0.5 equiv). The reaction was stirred for 10 min and warmed up to ambient temperature (22° C.). After stirring for another one hour, the reaction was complete and sat. NaHCO₃ solution was added to adjust the pH to 8, and the solvents were removed under high vacuum to give a solid as the crude intermediate.

The crude intermediate was dissolved in methanol (1.7 mL) and N,N-dimethylformaldehyde (0.2 mL), and the mixture was cooled to 0° C. Saturated K₂CO₃/MeOH solution (freshly prepared by stirring K₂CO₃ in MeOH overnight, 37 uL, 0.0083 mmol, 0.25 equiv). After 30 min, another batch of K₂CO₃/MeOH solution (144 uL, ˜0.033 mmol, 1.0 equiv) was added and the reaction was warmed up to ambient temperature (22° C.) and stirred for 20 h, and light yellow solid precipitated. The solid was filtered and washed with methanol (1.0 mL×3). The solid cake was dried and re-dissolved in DMSO (0.5 mL), and filtered through a cotton plug. The liquid was collected and dried under high vacuum to give desired 37 as a light yellow solid in high purity (4.4 mg, 41% over 2 steps).

37: Light yellow solid. ¹H NMR (500 MHz, CD₃SOCD₃) δ 8.43 (s, 1H), 8.22 (d, J=1.9 Hz, 1H), 7.15 (d, J=1.9 Hz, 1H), 7.09 (d, J=2.5 Hz, 1H), 4.69 (dt, J=11.3, 5.8 Hz, 1H), 3.93 (d, J=5.2 Hz, 1H), 3.70 (s, 1H), 2.64 (s, 3H), 2.58 (dd, J=13.0, 6.5 Hz, 1H), 2.15 (dd, J=13.0, 10.8 Hz, 1H); ¹³C NMR (125 MHz, CD₃SOCD₃) δ 158.4, 157.1, 136.1, 124.2, 123.2, 107.1, 93.4, 65.6, 60.4, 54.2, 39.9, 23.7; IR (thin film, cm¹) 3184, 1654, 1494, 1373, 1307; HRMS (ESI) calcd for C₁₂H₁₂N₅O₅[M−H]⁻ 306.0844, found 308.0846.

Cancer Cell Line Cytotoxicity Assays Cancer cell lines (MDA-MB-231, MCF7, Caco2, or U87) were plated on 96-well plates at 2,000 cells per well and incubated for 24 h at 37° C., 5% CO₂. AglA and derivatives were dissolved in DMSO, diluted to the appropriate concentrations, and the compound solutions or equivalent concentration of solvent (vehicle) were added to the plate and incubated for 72 h at 37° C., 5% CO₂. Following the manufacturer protocol, 20 μL of CellTiter 96® AQueous One Solution Cell Proliferation Assay (MTS, Promega), or CellTiter-Blue@ Cell Viability Assay (resazurin, Promega) was added. The plate was allowed to develop for 1-4 h at 37° C., 5% CO₂ and evaluated at 490 nm on an AccuSkan Go (Fisher Scientific) or at 560 nm excitation and 590 nm emission on a VarioSkan Lux Multimode Microplate Reader (Fisher Scientific).

While illustrative embodiments have been illustrated and described, it will be appreciated that various changes can be made therein without departing from the spirit and scope of the invention.

1. A compound of formula (I): 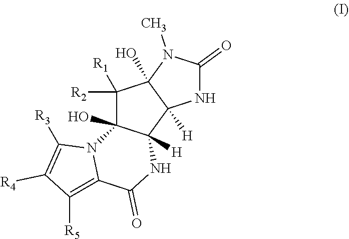
or a stereoisomer, racemate, or a pharmaceutically acceptable salt thereof,
wherein
R₁ is selected from the group consisting of H, F, Cl, and Br;
R₂ is selected from the group consisting of H, F, Cl, and Br;
R₃ is selected from the group consisting of Br, CF₃, SF₅, SO₂CF₃, SO₂CH₃, CN, and NO₂;
R₄ is selected from the group consisting of H, OH, F, Cl, Br, and CN; and
R₅ is H.

2. The compound of claim 1, wherein R₁, R₂, R₄, and R₅ are H and R₃ is Br.

3. The compound of claim 1, wherein R₁, R₂, and R₅ are H and R₃ and R₄ are Br.

4. The compound of claim 1, wherein R₁-R₅ are hydrogen.

5. The compound of claim 1, wherein R₁ and R₂ are independently selected from hydrogen and halo and R₃-R₅ are hydrogen.

6. A method for making a compound of formula (I), comprising converting a compound of formula (A) to a compound of formula (I) via a compound of formula (B): 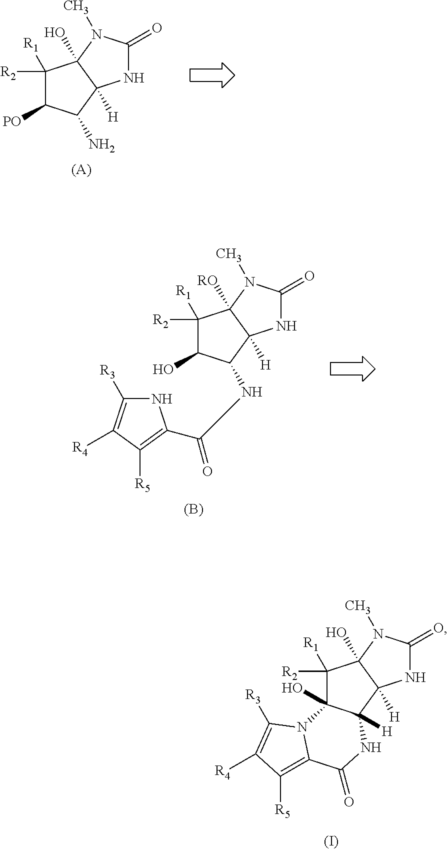
wherein the compound of formula (A) is reacted with 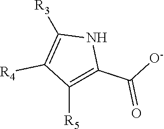
to provide amide (a): 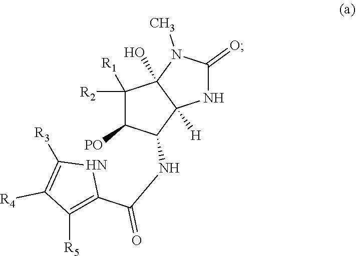
converting amide (a) to the compound of formula (B) followed by ring closure to provide 7-hydroxy compound (b): 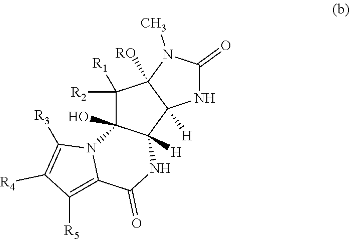
and
converting 7-hydroxy compound (b) to the compound of formula (I) by treatment aqueous acid,
wherein
P is an alcohol protecting group;
R is a methyl group;
R₁ is selected from the group consisting of H, F, Cl, and Br;
R₂ is selected from the group consisting of H, F, Cl, and Br;
R₃ is selected from the group consisting of Br, CF₃, SF₅, SO₂CF₃, SO₂CH₃, CN, and NO₂;
R₄ is selected from the group consisting of H, OH, F, Cl, Br, and CN; and
R₅ is H.

7. A pharmaceutical composition, comprising the compound of claim 1, or a stereoisomer, racemate, or pharmaceutically acceptable salt thereof, and a pharmaceutically acceptable carrier.

8. A method for treating a cancer, comprising administering a therapeutically effective amount of the compound of claim 1, or a stereoisomer, racemate, or pharmaceutically acceptable salt thereof, to a subject in need thereof.

9. The method of claim 8, wherein the cancer is breast cancer or glioblastoma.

10. A method for inhibiting protein synthesis through interactions with the peptidyl transferase center of a ribosome in a subject, comprising administering to a subject an effective amount of the compound of claim 1, or a stereoisomer, racemate, or pharmaceutically acceptable salt thereof, in an amount effective to inhibit protein synthesis through interactions with the peptidyl transferase center of a ribosome.

11. The method of claim 9, wherein the breast cancer is triple negative breast cancer or estrogen receptor positive breast cancer.
